SEQUENCE LISTING (5 sequences)

SEQ ID NO: 1 - DNA, 1701 bp, Artificial Sequence
  variant of hemagglutinin from Influenza A virus H3N2
atgaagaccatcattgctttgagctacattttctgtctggctctcggccaagaccttccaggaaatgacaacagcacagcaacgctgtgcctgggacatcatgcggtgccaaacggaacactagtgaaaacaatcacagatgatcagattgaagtgactaatgctactgagctagttcagaactcctcaacggggaaaatatgcaacaatcctcatcgaatccttgatggaataaactgcacactgatagatgctctattgggggaccctcattgtgatgtttttcaaaatgagaaatgggaccttttcgttgaacgcagcaaagctttcagcaactgttacccttatgatgtgccagattatgcctcccttaggtcactagttgcctcgtcaggcactctggagtttatcaatgagagtttcaattggactggggtcactcagaatgggacaagctcagcttgcaaaaggggacctaatagcagttttttcagtagactgaactggttgaccaaatcaggaagcacatatccagtgctgaacgtgactatgccaaacaatgacaattttgacaaactatacatttgggggattcaccacccgagcacgaaccaagaacaaaccagcctgtatgttcaagcatcagggagagtcacagtctctaccaggagaagccagcaaactataatcccgaatatcgggtccagaccctgggtaaggggtctgtctagtagaataagcatctattggacaatagttaagccgggagacgtactggtaattaatagtactgggaacctaatcgctcctcggggttatttcaaaatgcgcactgggaaaagctcaataatgaggtcagatgcacctattgatacctgtatttctgaatgcatcactccaaatggaagcattcccaatgacaagccctttcaaaacgtaaacaagatcacatatggagcatgccccaagtatgttaagcaaaacaccctgaagttggcaacagggatgcggaatgtaccagagaaacaaactagaggcctattcggcgcaatagcaggtttcatagaaaatggttgggagggaatgatagacggttggtacggtttcaggcatcaaaattctgagggcacaggacaagcagcagatcttaaaagcactcaagcagccatcgaccaaatcaatgggaaattgaacagggtaatcgagaagacgaacgagaaattccatcaaatcgaaaaggaattctcagaagtagaagggagaattcaggacctcgagaaatacgttgaagacactaaaatagatctctggtcttacaatgcggagcttcttgtcgctctggagaatcaacatacaattgacctgactgactcggaaatgaacaagctgtttgaaaaaacaaggaggcaactgagggaaaatgctgaagacatgggcaatggttgcttcaaaatataccacaaatgtgacaacgcttgcatagagtcaatcagaaatgggacttatgaccatgatgtatacagagacgaagcattaaacaaccggtttcagatcaaaggtgttgaactgaagtctggatacaaagactggatcctgtggatttcctttgccatatcatgctttttgctttgtgttgttttgctggggttcatcatgtgggcctgccagagaggcaacattaggtgcaacatttgcatttga

SEQ ID NO: 2 - DNA, 1677 bp, Artificial Sequence
  variant of hemagglutinin from Influenza A virus H3N2
atgaagaccatcattgctttgagctacattttctgtctggctctcggccaagaccttccaggaaatgacaacagcacagcaacgctgtgcctgggacatcatgcggtgccaaacggaacactagtgaaaacaatcacagatgatcagattgaagtgactaatgctactgagctagttcagagctcctcaacggggaaaatatgcaacaatcctcatcgaatccttgatggaatagactgcacactgatagatgctctattgggggaccctcattgtgatgtttttcaaaatgagacatgggaccttttcgttgaacgcagcaaagctttcagcaactgttttccttatgatgtgccagattatgcctcccttaggtcactagttgcctcgtcaggcactctggagtttatcactgagggtttcacttggactggggtcactcagaatgggggaagcaatgcttgcaaaaggggacctggtagcggttttttcagtagactgaactggttgaccaaatcaggaagcacatatccagtgctgaacgtgactatgccaaacaatgacaattttgacaaactatacatttgggggattttccacccgagcacgaaccaagagcaaaccagcgcgtatgttcaagcatcagggagagtcacagtctctaccaggagaagccagcaaactataatcccgaatatcgggtccagaagaataagcatctattggacaatagttaagccgggagacgtactggtaattaatagtaatgggaacctaatcgctcctcggggttatttcaaaatgcgcactgggaaaagctcaataatgaggtcagatgcacctattgatacctgtatttctgaatgcatcactccaaatggaagcattcccaatgacaagccctttcaaaacgtaaacaagatcacatatggagcatgccccaagtatgttaagcaaaacaccctgaagttggcaacagggatgcggaatgtaccagagaaacaaactagaggcctattcggcgcaatagcaggtttcatagaaaatggttgggagggaatgatagacggttggtacggtttcaggcatcaaaattctgagggcacaggacaagcagcagatcttaaaagcactcaagcagccatcgaccaaatcaatgggaaattgaacagggtaatcgagaagacgaacgagaaattccatcaaatcgaaaaggaattctcagaagtagaagggagaattcaggacctcgagaaatacgttgaagacactaaaatagatctctggtcttacaatgcggagcttcttgtcgctctggagaatcaacatacaattgacctgactgactcggaaatgaacaagctgtttgaaaaaacaaggaggcaactgagggaaaatgctgaagacatgggcaatggttgcttcaaaatataccacaaatgtgacaacgcttgcatagagtcaatcagaaatgggacttatgaccatgatgtatacagagacgaagcattaaacaaccggtttcagatcaaaggtgttgaactgaagtctggatacaaagactggatcctgtggatttcctttgccatatcatgctttttgctttgtgttgttttgctggggttcatcatgtgggcctgccagagaggcaacattaggtgcaacatttgcatttga

SEQ ID NO: 3 - DNA, 1677 bp, Artificial Sequence
  variant of hemagglutinin from Influenza A virus H3N2
atgaagaccatcattgctttgagctacattttctgtctggctctcggccaagaccttccaggaaatgacaacagcacagcaacgctgtgcctgggacatcatgcggtgccaaacggaacactagtgaaaacaatcacagatgatcagattgaagtgactaatgctactgagctagttcagaactcctcaacggggaaaatatgcaacaatcctcatcgaatccttgatggaataaactgcacactgatagatgctctattgggggaccctcattgtgatgtttttcaaaatgagaaatgggaccttttcgttgaacgcagcaaagctttcagcaactgttttccttatgatgtgccagattatgcctcccttaggtcactagttgcctcgtcaggcactctggagtttatcaatgagagtttcaattggactggggtcactcagaatgggacaagctcagcttgcaaaaggggacctaatagcagttttttcagtagactgaactggttgaccaaatcaggaagcacatatccagtgctgaacgtgactatgccaaacaatgacaattttgacaaactatacatttgggggattttccacccgagcacgaaccaagagcaaaccagcgcgtatgttcaagcatcagggagagtcacagtctctaccaggagaagccagcaaactataatcccgaatatcgggtccagaagaataagcatctattggacaatagttaagccgggagacgtactggtaattaatagtactgggaacctaatcgctcctcggggttatttcaaaatgcgcactgggaaaagctcaataatgaggtcagatgcacctattgatacctgtatttctgaatgcatcactccaaatggaagcattcccaatgacaagccctttcaaaacgtaaacaagatcacatatggagcatgccccaagtatgttaagcaaaacaccctgaagttggcaacagggatgcggaatgtaccagagaaacaaactagagaactattcggcgcaatagcaggtttcatagaaaatggttgggagggaatgatagacggttggtacggtttcaggcatcaaaattctgagggcacaggacaagcagcagatcttaaaagcactcaagcagccatcgaccaaatcaatgggaaattgaacagggtaatcgagaagacgaacgaggaattccatcaaatcgaaaaggaattctcagaagtagaagggagaattcaggacctcgagaaatacgttgaagacactaaaatagatctctggtcttacaatgcggagcttcttgtcgctctggagaatcaacatacaattgacctgactgactcggaaatgaacaagctgtttgaaaaaacaaggaggcaactgagggaaaatgctgaagacatgggcaatggttgcttcaaaatataccacaaatgtgacaacgcttgcatagagtcaatcagaaatgggacttatgaccatgatgtatacagagacgaagcattaaacaaccggtttcagatcaaaggtgttgaactgaagtctggatacaaagactggatcctgtggatttcctttgccatatcatgctttttgctttgtgttgttttgctggggttcatcatgtgggcctgccagagaggcaacattaggtgcaacatttgcatttga

SEQ ID NO: 4 - DNA, 1362 bp, Artificial Sequence
  variant of neuraminidase from Influenza A virus H3N2
atgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaagggggacctttaaggacagaagcccttatagggccttaatgagctgccctgtcggtgaagctccgtccccgtacaattcaaggtttgaatcggttgcttggtcagcaagtgcatgtcatgatggaatgggctggctaacaatcggaatttctggtccagatgatggagcagtggctgtattaaaatacaaccgcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcgatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggcaatggtgtttggataggaaggactaaaagtgacagttccagacatgggtttgagatgatttgggatcctaatggatggacagagactgatagtaggttctctatgagacaagatgttgtggcaataactaatcggtcagggtacagcggaagtttcgttcaacatcctgagctaacagggctagactgtatgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtag

SEQ ID NO: 5 - DNA, 1362 bp, Artificial Sequence
  variant of neuraminidase from Influenza A virus H3N2
atgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaaggaggacctttaaggacagaagcccttatagggccttaatgagctgccctgtcggtgaagctccgtccccgtacaattcaaggtttgaatcggttgcttggtcagcaagtgcatgtcatgatggaatgggctggctaacaatcggaatttctggtccagatgatggagcagtggctgtattaaaatacaaccgcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcgatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggcaatggtgtttggataggaaggactaaaagtgacagttccagacatgggtttgagatgatttgggatcctaatggatggacagagactgatagtaggttctctatgagacaagatgttgtggcaataactaatcggtcagggtacagcggaagtttcgttcaacatcctgagctaacagggctagactgtatgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtag

(19) United States
(12) Patent Application Publication
(10) Pub. No.: US 2016/0199481 A1
(43) Pub. Date: Jul. 14, 2016

(54) COMPOSITIONS AND METHODS FOR DIRECTED IMMUNOGEN EVOLUTION AND USES THEREOF

(71) Applicant: FRED HUTCHINSON CANCER RESEARCH CENTER, Seattle, WA (US)

(72) Inventor: Jesse D. Bloom, Seattle, WA (US)

(21) Appl. No.: 14/914,448

(22) PCT Filed: Aug. 21, 2014
(86) PCT No.: PCT/US2014/052180

§ 371 Date: Feb. 25, 2016

Related U.S. Application Data
(60) Provisional application No. 61/870,122, filed on Aug. 26, 2013.

(51) Int. Cl. A61K 39/145; C12N 15/10; C12N 7/00

(52) U.S. Cl. CPC A61K 39/145; C12N 7/00; C12N 15/1037; C12N 2760/16034; A61K 2039/525

(57) ABSTRACT

The present disclosure relates to compositions and methods for using a modified virus that infects a cell only if the virus presents a candidate antigen that binds with high affinity to a target antibody, thereby allowing for generation and identification of immunogens useful, for example, as vaccines.

### CROSS REFERENCE TO RELATED APPLICATION

This application claims the benefit of U.S. provisional patent application Serial Nos. 61/870,122, filed Aug. 26, 2013, which is incorporated herein by reference in its entirety.

### STATEMENT OF GOVERNMENT INTEREST

This invention was made with government support under Grant Nos. GM 102198-0 and AI093789-02 awarded by the National Institute of Health. The government may have certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_419WO_SEQUENCE_LISTING.txt. The text file is 10.7 KB, was created on Aug. 20, 2014, and is being submitted electronically via EFS-Web.

### BACKGROUND

1. Technical Field

The present disclosure relates to compositions and methods for generating antigens with greater specificity for target desirable antibodies and, more particularly, use of a modified virus presenting candidate antigens that can only infect a cell if a candidate antigen binds to a target antibody, which allows for rapid and high throughput selection for viruses that express antigens having high affinity for an antibody of interest.

2. Description of the Related Art

Vaccination has proven to be a tremendous public health tool, leading to the mitigation or even eradication of what were once some of the worst human diseases, such as smallpox, polio, and measles. However, many of the most problematic remaining pathogens have proved elusive targets for vaccines that elicit permanent protection. Viruses in this category include HIV, influenza, and hepatitis C. They share the feature that the immune system tends to produce antibodies that target rapidly evolving regions of viral proteins, allowing the viruses to readily escape (Burton et al., Proc. Nat'l. Acad. Sci. USA 102:14943, 2005). It is therefore exciting that new tools have begun to isolate broadly neutralizing antibodies against these viruses from the serum of infected or vaccinated individuals (Law et al., Nat. Med. 14:25, 2008; Walker et al., Science 326:285, 2009; Sui et al., Nat. Struct. Mol. Biol. 16:265, 2009). These antibodies provide proof of principle that it may be possible to create vaccines against these rapidly evolving pathogens. Unfortunately, a workable strategy for actually creating such vaccines has not yet been developed.

Instead, current approaches rely on administering such antibodies directly with passive injections (Brekke and Sandlie, Nat. Rev. Drug Discov. 2:52, 2003), or more speculatively introducing them by gene therapy (Balazs et al., Nat. Biotechnol. 31:647, 2013; Balazs et al., Nature 481:81, 2011). However, such approaches are costly and untested, and lack many of the advantages of vaccination. Specifically, vaccination induces the immune system to do most of the work, and often requires just one or a few cheap and easy administrations to provide lifelong protection. Thus, there is a need in the art for alternative methods for eliciting an immune response against a variety of diseases and conditions (e.g., infection, cancer, inflammation). The present disclosure meets such needs, and further provides other related advantages.

### BRIEF DESCRIPTION THE DRAWINGS

FIGS. 1A-1C shows images of HA crystal structures. Attempted growth of a virus with extensive mutations in the HA receptor-binding pocket selects for a mutation near the active site of NA. (A) Crystal structure (PDB 4HMG) of an HA monomer with a sialic-acid analogue (purple spheres) bound in the receptor-binding pocket. The sites of the binding-pocket mutations are shown in colors other than gray, and the site of stalk mutation K62E in HA2 is also indicated. (B) Zoomed-in image of the receptor-binding pocket of the HA structure shown in (A). (C) Crystal structure (PDB 2HU4) of an NA monomer with oseltamivir (green spheres) in the active site and the site of the passage-derived G147R mutation shown in red.

FIG. 2 shows that viruses with the HA receptor-binding mutations can only be rescued with the mutant G147R NA. Shown are viral titers in the supernatant 72 hours after attempted rescue of the indicated viruses by reverse genetics. Virus containing the BindMut HA can only be rescued in combination with the G147R NA. Further passage of this BindMut HA/G147R NA virus selected for the additional K62E mutation in HA2. The PassMut HA (which contains this HA2 mutation) also can only be rescued in combination with the G147R NA. Shown are the mean and standard errors for three replicates.

FIGS. 3A-3D show HA is still required for viral membrane fusion. (A) Introduction of the fusionblocking G1E mutation into WT HA does not substantially impact HA surface expression, as quantified by antibody staining and flow cytometry of transfected 293T cells. (B) Introduction of the G1E mutation into PassMut HA also does not substantially impact HA surface expression. (C) G1E completely blocks the rescue of infectious virus by reverse genetics, regardless of the NA used. Shown are the viral titers in the supernatant 70 hours after attempted rescue of the indicated viruses by reverse genetics. (D) Infectivity of all viral variants is neutralized by the fusion-inhibiting antibody FI6v3, regardless of which glycoprotein the virus uses to bind to the receptor. In all panels, data represent the mean and standard errors of three replicates.

FIGS. 4A-4D show that the G147R NA is an active sialidase that is inhibited by oseltamivir. (A) Surface expression of WT and G147R NA with C-terminal V5 epitope tags in transfected 293T cells. Expression of G147R NA is approximately 70% that of WT. (B) Rate of MUNANA cleavage at increasing substrate concentrations. Michaelis-Menten kinetics curves were fit to determine KM and Vmax. (C) Enzyme kinetics for WT and G147R NA. Vmax is also normalized to expression levels in (A) to give a value proportional to kcat. (D) NA activity at increasing concentrations of oseltamivir. Both NAs are inhibited at similar concentrations. The y-axis shows the percent remaining activity relative to the same NA variant in the absence of oseltamivir. For all panels, data represent the mean and standard error of three replicates.

FIGS. 5A-5D show that oseltamivir neutralizes and inhibits hemagglutination by viruses that utilize G147R NA as the receptor-binding protein. (A) The extent of virus neutralization by oseltamivir depends on the degree to which NA is utilized as the receptor-binding protein. PassMut HA/G147R NA uses NA as the receptor-binding protein, and is nearly completely neutralized by oseltamivir. WT HA/G147R NA uses both HA and NA as receptor-binding proteins, and is partially neutralized by oseltamivir. WT HA/WT NA uses HA as the receptor-binding protein, and is resistant to neutralization by oseltamivir. (B) Similar effects as in (A) are seen when viral infectivity is inhibited with polyclonal anti-NA antibodies from mouse serum. The plots show neutralization by serum from mice infected with virus carrying the G147R NA, or mock infected with PBS. Both (A) and (B) represent the mean and standard error of three replicates. (C) Agglutination of red blood cells (RBCs) by PassMut HA/G147R NA is inhibited by oseltamivir while WT HA/WT NA and WT HA/G147R NA are resistant to inhibition at all concentrations tested. RBCs from the indicated species were incubated with 8 HA units of virus pretreated with the indicated amount of oseltamivir. (D) Agglutination of turkey RBCs by PassMut HA/G147R NA is inhibited at low concentrations of polyclonal anti-NA antibodies from mouse serum. WT HA/WT NA and WT HA/G147R NA are much more resistant to inhibition. Values are reported as the reciprocal of the dilution factor for which complete inhibition was seen.

FIGS. 6A and 6B show that G147R NA-only virus-like particles (VLPs) agglutinate red blood cells, and agglutination is inhibited by oseltamivir. (A) A hemagglutination assay was performed using WT and G147R NA VLPs. VLPs were serially diluted two-fold across a U-bottom plate, turkey red blood cells (RBCs) were added, and the plate was imaged every 20 minutes. At 60 minutes, oseltamivir was added to all wells to a final concentration of 10 nM. The plate was imaged again 20 minutes later by which time agglutination by G147RVLPs had been reversed. (B) A hemagglutination inhibition assay was performed using serial three-fold dilutions of oseltamivir across a U-bottom plate. VLPs from HA assay were added at a concentration corresponding to the 1:8 dilution in (A). The plate was imaged 60 minutes after the addition of turkey RBCs. Oseltamivir was then added to all wells at a final concentration of 1.6 nM and plate was imaged 20 minutes later.

FIGS. 7A-7C show that treatment with an exogenous bacterial sialidase (receptor-destroying enzyme, RDE) inhibits viruses expressing wild-type HA and non-binding NA (A), but only partially inhibits infection by receptor-binding NA viruses (B with wild type HA present; C with mutant HA present). The receptor-binding NA virus having only a mutant HA present is inhibited more than when wild type HA is present. Cells both with and without RDE pre-treatment were infected with each viral variants at an MOI of 0.05. Supernatant was collected every 12 hours post-infection and viral titers determined. Data represent the mean and standard error of three replicates.

FIG. 8 shows the directed immunogen evolution scheme of this disclosure. The top box shows why desirable antibodies are often elicited only at low levels—most of the antigen binds to other B-cells, and B-cells expressing the desirable antibody are only activated at low levels. To engineer an antigen that activates these target B cells more strongly, we create an engineered cell line expressing the target antibody on its surface. We then put a candidate epitope in the top of the influenza viral HA. These viruses are grown using the receptor-binding NA, which is then blocked with oseltamivir. These viruses are now unable to enter normal cells, as neither their HA nor NA can bind to the cell. However, if the epitope binds strongly the antibody expressed on the engineered cell, the virus can infect the cells. Repeated passages of virus mutant libraries in these engineered cells selects for variants that bind strongly to the target antibody. These viruses are then candidate vaccine immunogens.

FIG. 9 shows that viruses dependent on the receptor-binding NA are inhibited by the addition of oseltamivir. The PassMut HA/G147R NA virus uses NA as the predominant receptor-binding protein, and is nearly completely neutralized by oseltamivir. The WT HA/G147R NA virus uses both HA and NA as receptor-binding proteins, and is partially neutralized by oseltamivir. The WT HA/WT NA virus uses HA as the receptor-binding protein, and is resistant to neutralization by oseltamivir.

FIGS. 10A and 10B show that (A) constructs developed that allow expression of membrane bound antibody in an IgM form (mIgM) on the surface of cell lines such as 293T and MDCK cells. The construct consists of a CMV promoter driving expression of the antibody light chain followed by a 2A linker and the antibody heavy chain in an IgM form. At the C-terminus is the transmembrane domain from the mouse B7.1 protein. There is a V5 epitope tag at the end of the light chain. (B) The plots show flow cytometry staining (anti-V5 antibody) of untransduced plain cells, or cells transduced with membrane bound antibodies P20.1 or aMyc. The transduced cells express clearly detectable levels of antibody on their surface.

FIG. 11 shows the proof of principle of the directed immunogen evolution scheme of this disclosure. Influenza HA was engineered to express the P4 epitope tag at the top of the molecule. Viruses carrying these epitope HAs (these viruses are named EP5 viruses in the plot) as well as no-epitope control viruses were grown to high titers using the receptor-binding NA. These viruses were then used to express normal cells (no mIgM) or cells expressing the P20.1 antibody against the P4 tag (P20.1 mIgM). In the absence of oseltamivir and RDE, all viruses could infect all cells by virtue of the receptor-binding NA. But upon addition of oseltamivir and RDE, the receptor-binding NA was neutralized. The only combinations that could then grow involved viruses carrying the epitope tag (EP5) with cells expressing the cognate antibody (P20.1). This provides a proof-of-principle that membrane antibodies can be used to selectively grow viral variants that bind strongly to the target antibodies.

### DETAILED DESCRIPTION

In certain aspects, the present disclosure provides compositions and methods for reverse engineering epitopes that can then be used to elicit desirable antibodies. As described herein, a recombinant Orthomyxoviridae virus having alterations to the hemagglutinin (HA) and neuraminidase (NA) proteins is used, which allows one to chemically (e.g., small molecule, antibody) switch on or off the viral replication cycle. Moreover, these modified viruses can accommodate the addition of a random library of antigens (epitopes) at the most antigenic location on the virus—the globular head of the HA protein. For example, in the presence of the small molecule inhibitor, the virus cannot infect normal cells, but when the virus carries an antigen that binds to a target antibody or other binding domain expressed by a host cell, the virus will attach to and infect the cells through the antigen-binding domain interaction. This scheme can, therefore, be used as rapid selection for viruses that express antigens with high affinity for a target binding domain. Furthermore, candidate antigens can rapidly be improved since the viruses naturally have a high mutation rate. Alternatively, or in addition, candidate antigens or naturally mutated variants can be improved by introducing mutations by mutagenesis (random or directed).

In another aspect, a virus identified as having a strong ability to infect cells through the HA-epitope fusion antigen interaction with a cell binding domain, the virus can be used directly as a vaccination agent. Thus, another advantage of the compositions and methods of the instant disclosure is that the recombinant virions are naturally highly immunogenic and can be grown to large titers, so the same viral vector used to select for the antigen can also be used deliver it to a host cheaply and efficiently.

In certain embodiments, recombinant Orthomyxoviridae virions comprising a modified genome encoding (i) a non-binding hemagglutinin (HA^{nb}) variant, (ii) a fusion protein of a HA^{nb} with an epitope, or (iii) a fusion protein of a HA^{nb} with an exogenous binding domain (EBD); and encoding a variant neuramindase (NA^{b}) protein capable of binding to a target cell, wherein each Orthomyxoviridae virion displays on its surface the NA^{b} protein with a HA^{nb} protein, the NA^{b} protein with a HA^{nb}-epitope fusion protein, or a the NA^{b} protein with a HA^{nb}-EBD fusion protein, respectively. For example, the Orthomyxoviridae virion displaying on its surface the NA^{b} protein with a HA^{nb} protein may be used as a vaccine to generate antibodies against the HA stalk region (i.e., fusion domain). Similarly, the Orthomyxoviridae virion displaying on its surface the NA^{b} protein with a HA^{nb}-epitope fusion protein can be used as a vaccine to generate antibodies (e.g., neutralizing antibodies) against a preferred epitope. Furthermore, the Orthomyxoviridae virion displaying on its surface the NA^{b} protein with a HA^{nb}-epitope may be used to rapidly “evolve” the epitopes so that they have higher affinity for an antibody or other binding domain of interest.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives or enumerated components. As used herein, the terms “include,” “have” and “comprise” are used synonymously, which terms and variants thereof are intended to be construed as non-limiting.

As used herein, “nucleic acid” or “nucleic acid molecule” refers to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in sugar moieties or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety may be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids” (PNAs), which comprise naturally occurring or modified nucleic acid bases attached to a polyamide backbone. Nucleic acid molecules can be either single stranded or double stranded.

Further, an “isolated nucleic acid molecule” refers to a polynucleotide molecule in the form of a separate fragment or as a component of a larger nucleic acid construct, which has been separated from its source cell (including the chromosome it normally resides in) at least once in a substantially pure form. For example, a DNA molecule that encodes a recombinant polypeptide, peptide, or variant thereof, which has been separated from a cell or from the genomic DNA of a cell, is an isolated nucleic acid molecule. Another example of an isolated nucleic acid molecule is a bacteriophage promoter (e.g., T5 or T7), or nucleic acid expression control sequence, which can be cloned into a vector capable of replication in a suitable host cell. Still another example of an isolated nucleic acid molecule is a chemically synthesized or PCR synthesized nucleic acid molecule.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In other embodiments, a mutation is a substitution of one or more nucleotides or residues.

A “binding domain” or “binding region,” as used herein, refers to a protein, polypeptide, oligopeptide, peptide, a saccharide, a polysaccharide, nucleic acid molecules or other biological molecule that possesses the ability to specifically recognize and bind to a target (e.g., epitope, HA, NA). A binding domain includes any naturally occurring, synthetic, semi-synthetic, or recombinantly produced binding partner for a biological molecule or another target of interest. Exemplary binding domains include single chain antibody variable regions (e.g., domain antibodies, sFv, single chain Fv fragment (scFv), Vα/Vβ single-chain TCR (scTv), Fab, F(ab′)₂, receptor ectodomains (e.g., TNF), or ligands (e.g., cytokines, chemokines). In certain embodiments, a neuramindase variant capable of binding to an “acceptor” molecule (NA^{b}) has a binding domain, and the acceptor may be sialic acid, another receptor molecule, or a combination thereof. A variety of assays are known for identifying binding domains of the present disclosure that specifically bind a particular target, including Western blot, ELISA, and Biacore® analysis. Exemplary binding domains comprise immunoglobulin light and heavy chain variable domains (e.g., scFv, Fab) and are herein referred to as “immunoglobulin binding domains” or “immunoglobulin binding proteins.” Immunoglobulin binding domains can be incorporated into a variety of protein scaffolds or structures as described herein, such as an antibody or an antigen binding fragment thereof, a scFv-Fc fusion protein, a chimeric antigen receptor, or a fusion protein comprising two or more of such immunoglobulin binding domains.

A binding domain and a fusion protein thereof “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (KO of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al., Ann. N.Y. Acad. Sci. 51:660, 1949; and U.S. Pat. Nos. 5,283,173, 5,468,614, or the equivalent).

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds, as well as an antigen-binding portion of an intact antibody that has or retains the capacity to bind a target molecule. A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

As used herein, a protein domain (e.g., a binding domain, HA fusion (stalk) domain, HA globular head region, an Fc region constant domain portion) or a protein (which may have one or more domains) “consists essentially of” a particular amino acid sequence when the amino acid sequence of a protein domain or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of the domain or protein and do not substantially affect (i.e., do not reduce the activity by more than 50%, such as no more than 40%, 30%, 25%, 20%, 15%, 10%, or 5%) the activity of the domain(s) or protein (e.g., the binding or fusion activity of a HA variant or the target binding affinity of a binding protein or epitope).

A “receptor” is a protein molecule, present in the plasma membrane or in the cytoplasm of a cell or released from a cell membrane, to which a signal molecule (i.e., a ligand, such as a hormone, a neurotransmitter, a toxin, a cytokine) may attach or bind. The binding of a ligand to a receptor can result in a conformational change that will ordinarily initiate a cellular response, but some ligands merely block receptors without inducing any response (e.g., antagonists). Some receptor proteins are peripheral membrane proteins, also known as transmembrane proteins, which often have an extracellular domain (ECD), a transmembrane domain, and a cytoplasmic domain. Some cell membrane receptors may be cleaved and the released ECD can still bind its target or be involved in biological signaling or both. Other receptors are intracellular proteins, such as those for steroid and intracrine peptide hormone receptors.

As used herein, an “Orthomyxoviridae library” refers to a collection of nucleic acid molecule sequences or fragments that may be incorporated into a viral vector, which may be further replicated on an appropriate host cell. The target nucleic acid molecules of this disclosure may be introduced into a variety of different hemagglutinin (HA) variants, such as HA mutants that can no longer bind to its native cell surface receptor, sialic acid sugars and may have a partial or complete globular head deletion, wherein the deleted non-binding HA (HA^{nb}) protein maintains a functional fusion domain.

The term “construct” refers to any polynucleotide that contains a recombinant nucleic acid. A construct may be present in a vector (e.g., a bacterial vector, a viral vector) or may be integrated in a genome, for example. A “vector” is a nucleic acid molecule that is capable of transporting another nucleic acid. Vectors may be, for example, plasmids, cosmids, viruses, or phage.

The term “operably-linked” refers to the association of nucleic acid sequences on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself. In the present specification, “plasmid,” “expression plasmid,” and “vector” are often used interchangeably as the plasmid is the most commonly used form of vector at present. However, this disclosure is intended to include such other forms of expression vectors that serve equivalent functions and which are, or become, known in the art.

The term “expression”, as used herein, refers to the process by which a polypeptide is produced based on the nucleic acid sequence of a gene. The process includes both transcription and translation.

The term “introduced” in the context of inserting a nucleic acid sequence into a cell, means “transfection” or ‘transformation” or “transduction” and includes reference to the incorporation of a nucleic acid sequence into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed (e.g., transfected mRNA).

As used herein, the term “isolated” refers to a substance that has been removed from the source in which it naturally occurs. A substance need not be purified in order to be isolated. For example, a protein produced in a host cell is considered isolated when it is removed or released from the cell. A protein contained within a crude cell lysate fraction is considered “isolated” for purposes of the present disclosure. Further, an “isolated nucleic acid molecule” refers to a polynucleotide molecule in the form of a separate fragment or as a component of a larger nucleic acid construct, which has been separated from its source cell, including the chromosome it normally resides in, at least once. For example, a DNA molecule that encodes a recombinant polypeptide, peptide, or variant thereof, which has been separated from the genomic DNA of a cell, is an isolated DNA molecule.

As used herein, the term “purified” refers to a substance that has been rendered at least partially free of contaminants and other materials that typically accompany it. Substances can be purified to varying degrees. A substance is “substantially pure” when a preparation or composition of the substance contains less than about 1% contaminants. A substance is “essentially pure” when a preparation or composition of the substance contains less than about 5% contaminants. A substance is “pure” when a preparation or composition of the substance contains less than about 2% contaminants. For substances that are “purified to homogeneity,” contaminants cannot be detected with conventional analytical methods.

The term “recombinant” refers to a polynucleotide or polypeptide that does not naturally occur in a virus or host cell. A recombinant molecule may contain two or more naturally-occurring sequences that are linked together in a way that does not occur naturally. A recombinant cell contains a recombinant polynucleotide or polypeptide.

As used herein, the terms “percent sequence identity,” “percent identity,” and “% identity” refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence in order to effect optimal alignment. Percent identity is calculated by dividing the number of matched portions in the comparison window by the total number of positions in the comparison window, and multiplying by 100. The number of matched positions in the comparison window is the sum of the number of positions of the comparison polynucleotide or polypeptide in the window that are identical in sequence to the reference polynucleotide or polypeptide and the number of positions of the reference polynucleotide or polypeptide in the comparison window that align with a gap in the comparison polynucleotide or polypeptide. Determination of optimal alignment and percent sequence identity is performed using the BLAST and BLAST 2.0 algorithms (see, e.g., Altschul et al., J. Mol. Biol. 215:403, 1990; Altschul et al., Nucleic Acids Res. 25:3389, 1997). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, Proc. Nat'l Acad. Sci. USA 89:10915, 1989). In a preferred embodiment, BLAST algorithm parameters set a default parameters are used to identify percent identity of a target nucleic acid molecule or a target polypeptide molecule as compared to a reference nucleic acid molecule or a reference polypeptide molecule, respectively.

Optimal alignment of sequences for comparison can be conducted, e.g., by the local homology algorithm of Smith and Waterman, Adv. Appl. Math. 2:482, 1981; by the homology alignment algorithm of Needleman and Wunsch, J. Mol. Biol. 48:443, 1970; by the search for similarity method of Pearson and Lipman, Proc. Nat'l Acad. Sci. USA 85:2444, 1988; by computerized implementations of these algorithms (GAP, BESTFIT, FASTA, and TFASTA in the GCG Wisconsin Software Package); or by visual inspection (see, generally, Current Protocols in Molecular Biology, F. M. Ausubel et al., eds., Current Protocols, a joint venture between Greene Publishing Associates, Inc. and John Wiley & Sons, Inc., 1995 Supplement).

As used herein, the term “reference sequence” refers to a specified sequence to which another sequence is compared. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity. The term “reference sequence” is not intended to be limited to wild-type sequences, and can include engineered, variant, or altered sequences.

The term “biological sample” includes a blood sample, biopsy specimen, tissue explant, organ culture, biological fluid (e.g., blood, serum, urine, CSF) or any other tissue or cell or other preparation from a subject or a biological source. A subject or biological source may, for example, be a human or non-human animal, a primary cell culture or culture adapted cell line including genetically engineered cell lines that may contain chromosomally integrated or episomal recombinant nucleic acid sequences, somatic cell hybrid cell lines, immortalized or immortalizable cell lines, differentiated or differentiatable cell lines, transformed cell lines, or the like. In further embodiments of this disclosure, a subject or biological source may be suspected of having or being at risk for having a disease, disorder or condition, including a malignant disease, disorder or condition or a viral infection. In certain embodiments, a subject or biological source may be suspected of having or being at risk for having a hyperproliferative, inflammatory, autoimmune or infectious disease, and in certain other embodiments of this disclosure the subject or biological source may be known to be free of a risk or presence of such disease, disorder, or condition.

“Treatment,” “treating” or “ameliorating” refers to either a therapeutic treatment or prophylactic/preventative treatment (e.g., vaccine). A treatment is therapeutic if at least one symptom of disease in an individual receiving treatment improves or a treatment may delay worsening of a progressive disease in an individual (e.g., by eliciting an immune response), or prevent onset of additional associated diseases.

A “therapeutically effective amount (or dose)” or “effective amount (or dose)” of a specific binding molecule, compound, or virus refers to that amount of the compound or virus sufficient to result in amelioration of one or more symptoms of the disease being treated in a statistically significant manner or eliciting an immune response. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously.

The term “pharmaceutically acceptable” refers to molecular entities and compositions that do not produce allergic or other serious adverse reactions when administered using routes well known in the art.

A “patient in need” refers to a patient at risk of, or suffering from, a disease, disorder or condition that is amenable to treatment or amelioration with a Orthomyxoviridae virion or a composition thereof provided herein to elicit an immune response (e.g., function as a vaccine).

In certain embodiments, the instant disclosure provides recombinant Orthomyxoviridae virions comprising a modified genome encoding a fusion protein of a HA^{nb} with an epitope, and encoding a variant neuramindase (NA^{b}) protein capable of binding to a target cell, wherein the Orthomyxoviridae virion displays on its surface the NA^{b} protein with a HA^{nb}-epitope fusion protein. In further embodiments, a method for eliciting an immune response against an epitope comprises administering the aforementioned recombinant Orthomyxoviridae virion to a subject (e.g., human), wherein such immune response is an antibody specific for the epitope.

In certain other embodiments, the instant disclosure provides recombinant Orthomyxoviridae virions comprising a modified genome encoding a fusion protein of a HA^{nb} with an exogenous binding domain (EBD); and encoding a variant neuramindase (NA^{b}) protein capable of binding to a target cell, wherein the Orthomyxoviridae virion displays on its surface the NA^{b} protein with a HA^{nb}-EBD fusion protein.

In further embodiments, the instant disclosure provides recombinant Orthomyxoviridae virions comprising a modified genome encoding a non-binding hemagglutinin (HA^{nb}) variant and encoding a variant neuramindase (NA^{b}) protein capable of binding to a target cell, wherein the Orthomyxoviridae virion displays on its surface the NA^{b} protein with a HA^{nb} protein. In further embodiments, a method for eliciting an immune response against a hemagglutinin stalk region by administering the aforementioned recombinant Orthomyxoviridae virion to a subject (e.g., human), wherein the immune response is an antibody specific for the epitope, such as a neutralizing antibody.

In still further embodiments, the instant disclosure provides a library of recombinant Orthomyxoviridae comprising a plurality of Orthomyxoviridae virions having a modified genome encoding a fusion protein of a non-binding hemagglutinin (HA^{nb}) variant with an epitope or an exogenous binding domain (EBD), and a variant neuramindase (NA^{b}) capable of binding to a target cell, whereby the virions collectively comprise a library of nucleic acid molecules encoding a population of different epitopes, each member of the epitope population capable of being expressed as a HA^{nb}-epitope fusion protein on the surface of the virion, and whereby blocking NA^{b} binding to a target cell inhibits viral replication and binding of a HA^{nb}-epitope fusion protein to a target cell promotes viral replication.

In yet further embodiments, the instant disclosure provides a method for identifying or evolving an epitope or EBD by (a) contacting a cell with an inhibitor of a variant neuramindase (NA^{b}) protein binding to an acceptor molecule on the cell and a population of Orthomyxoviridae virions comprising a modified genome containing a nucleic acid molecule that encodes a non-binding hemagglutinin variant (HA^{nb})-epitope fusion protein or HA^{nb}-EBD fusion protein, and encoding an NA^{b} protein capable of binding to an acceptor molecule on the cell, wherein each virion displays at its surface a HA^{nb} fusion protein and the population of epitopes have a range of binding specificities, wherein at least one epitope in the population of epitopes is capable of specifically binding a target molecule on the cell that is not the neuraminidase acceptor molecule and is capable of promoting viral replication; and (b) detecting a virion that replicates on the cells in the presence of the inhibitor of NA^{b} protein binding, thereby identifying an epitope with a desired specificity for the target molecule. In certain embodiments, the identified epitope or EBD is further mutated (naturally, random, or directed) to enhance binding.

In certain embodiments, the instant disclosure provides a plurality of recombinant nucleic acid molecules, comprising a plurality of vectors that individually include a nucleic acid molecule that encodes a PB1 RNA polymerase protein, a PB2 RNA polymerase protein, a PA RNA polymerase protein, a nucleoprotein (NP), a matrix (M) protein, a non-structural (NS) protein, a non-binding hemagglutinin (HA^{nb}) variant fusion protein (fused with an epitope or EBD), and a variant neuramindase (NA^{b}) protein capable of binding to a target cell, wherein the recombinant nucleic acid molecules are expressed when introduced into a host cell and the host cell is capable of producing Orthomyxoviridae virions that display on the virion surface the HA^{nb} fusion protein and NA^{b} protein.

In certain embodiments, the instant disclosure provides a recombinant cell, comprising a plurality of nucleic acid molecules that individually encode a PB1 RNA polymerase protein, a PB2 RNA polymerase protein, a PA RNA polymerase protein, a nucleoprotein (NP), a matrix (M) protein, a non-structural (NS) protein, a non-binding hemagglutinin (HA^{nb}) variant fusion protein (fused to an epitope or EBD), and a variant neuramindase (NA^{b}) protein capable of binding to a target cell, wherein the cells are capable of producing Orthomyxoviridae virions that display on the virion surface the HA^{nb} fusion protein and the NA^{b} protein.

In certain embodiments, any of the aforementioned recombinant Orthomyxoviridae virions are capable of replicating on a host cell. In certain embodiments, the Orthomyxoviridae virion genome comprises a truncated PB1 coding sequence comprising about 80 coding nucleotides of the PB1 5′-terminus and about 80 coding nucleotides of the PB1 3′-terminus flanking and fused to a reporter molecule, such as green fluorescent protein, enhanced green fluorescent protein (eGFP), red fluorescent protein, luciferase, aequorin, β-galactosidase, or alkaline phosphatase. In still other embodiments, the about 80 coding nucleotides of the PB1 5′-terminus comprise mutations at each potential start codon. In further embodiments, the PB1-reporter molecule fusion protein is PB1flank-eGFP or PB1-mCherry (see Bloom et al., Science 328:1272, 2010, which PB1 flank constructs are incorporated herein in their entirety).

In certain embodiments, any of the aforementioned compositions or methods are provided wherein the HA^{nb} protein comprises a partial or complete globular head deletion, and the deleted HA^{nb} protein maintains a functional fusion domain. In certain embodiments, the HA^{nb} protein comprises a deletion ranging from about 10 amino acids to all amino acid residues at position 53 to 276 based on wild-type amino acid sequence numbering of influenza A subtype 3 hemagglutinin. An exemplary HA^{nb} comprises a deletion mutation of amino acid residues 221 to 228 based on wild-type amino acid sequence numbering of influenza A subtype 3 hemagglutinin. In further embodiments, the HA^{nb} protein further comprises a substitution mutation at position 98, 183, 194, or any combination thereof based on numbering of the wild type amino acid sequence of influenza A subtype 3 hemagglutinin, such as the substitution mutations of Y98F, H183F, L194A, or any combination thereof. In still further embodiments, the He fusion protein further comprises one to ten mutations that add glycosylation sites, such as substitution mutations at position 45, 63, 83, 122, 124, 126, 135, 144, 146, 248, or any combination thereof based on numbering of the wild type amino acid sequence of influenza A subtype 3 HA and more specifically the substitution mutations are S45N, D63N, T83K, T122N, G124S, T126N, G135T, G144N, G146S, N248T, or any combination thereof.

In certain embodiments, any of the aforementioned compositions or methods are provided wherein the NA^{b} protein comprises a substitution mutation at position 147 based on numbering of the wild type amino acid sequence of influenza A subtype 2 neuraminidase. In further embodiments, an inhibitor of NA^{b} binding is an antibody specific for NA or a small molecule, such as oseltamivir.

In certain embodiments, any of the aforementioned compositions or methods are provided wherein the virion is based on an Influenzavirus (e.g., A, B, C), Isavirus, Thogotovirus (e.g., Thogoto virus, a Dhori virus), Quaranfil virus, a Johnston Atoll virus, a Lake Chad virus, or a Cygnet River virus. In further embodiments, the Influenzavirus is an influenza A virus, influenza B virus, or influenza C virus. In still further embodiments, the virion is based on an influenza A virus subtype comprising any combination of hemagglutinin and neuramindase subtypes, wherein the hemagglutinin subtype is selected from H1 to H17 and the neuramindase subtype is selected from N1 to N10, such as influenza A virus subtypes H1N1, H1N2, H2N2, H3N2, H5N1, H5N2, H7N2, H7N3, H7N7, H9N2, or H10N7.

In certain embodiments, any of the aforementioned compositions or methods are provided wherein the epitope comprises from eight to about 500 amino acids and may be an epitope that is specific for a known antibody, cell surface receptor (e.g., chimeric antigen receptor), or cell surface protein. In further embodiments, the exogenous binding domain is a single chain antibody variable region, a single chain T cell receptor variable region, a receptor ectodomain, or a ligand. Exemplary single chain antibody variable region include domain antibodies, sFv, scTv, scFv, F(ab′)₂, or Fab.

### EXAMPLES

### Example 1

### Construction of Influenza NA Receptor Binding Mutant

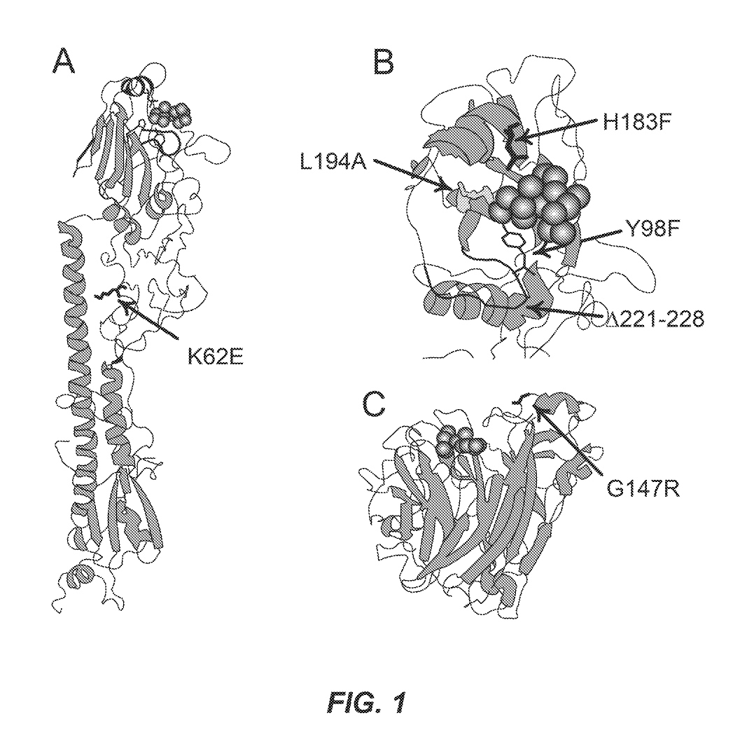

A hemagglutanin (HA) gene from influenza A/Hong Kong/2/1968 (H3N2) strain was mutated to eliminate its sialic-acid receptor binding activity. In the H3 numbering scheme, these mutations included Y98F, H183F, L194A, and deletion of amino acids 221 to 228 (FIGS. 1A and B). These mutations were chosen because the three point mutations were previously shown to individually nearly abolish HA receptor binding (Martin et al., Virology 241:101, 1998), and the loop deletion is near the HA receptor binding pocket (Yang et al., PLoS pathogens 6:e1001081, 2010). In addition, seven N-linked glycosylation site motifs were added at positions where glycosylation is found in contemporary human H3N2 HA proteins (i.e., potentially glycosylated asparagines at residues 45, 63, 122, 126, 133, 144, and 246 in H3 numbering), since glycosylation of HA has been shown to reduce receptor avidity (Das et al., Proc. Nat'l Acad. Sci. U.S.A. 108:E1417). This presumed binding-deficient mutant HA is referred to as BindMut HA.

The BindMut HA was used as a negative control during rescue of viruses by reverse genetics (Bloom et al., Science 328:1272, 2010) for a series of other experiments. Growth of influenza A/WSN/33 (H1N1) strain containing the BindMut HA was not expected due to its presumed lack of receptor-binding ability. Surprisingly, in one rare instance, a virus with the BindMut HA that grew to moderate titers in tissue culture was isolated. The isolated virus contained the BindMut HA and all of the other genes from the A/WSN/33 (H1N1) strain. Sequencing of this isolate showed no mutations or reversions in HA, but one point mutation was identified in neuraminidase (NA), G147R (in N2 numbering scheme). This mutation is located somewhat above the NA active site as shown in the NA crystal structure (FIG. 1C). Further passage of the virus yielded a further variant that grew to increased titers. This virus had retained the G147R NA mutation, and had also acquired an HA stalk mutation in the HA2 subunit, K62E (in H3 numbering scheme) (FIG. 1A). This HA mutant variant, which contains all of the original receptor-binding site mutations and glycosylation sites plus the K62E stalk mutation, is henceforth referred to as PassMut HA (Passage Mutant HA).

To determine whether the HA and NA mutations were responsible for the growth phenotypes observed, reverse-genetics plasmids for all HA and NA variants were created. Three HA variants were made: a variant we will term wild-type (WT) HA which has the seven glycosylation sites added but none of the receptor-binding site mutations, the BindMut HA, and the PassMut HA. Two NA variants were created: Wild-type A/WSN/33 NA (WT NA), and WSN NA with the G147R mutation (NA^{G147R}). The rescue of viruses containing all combinations of these HAs and NAs in the WSN background was then examined.

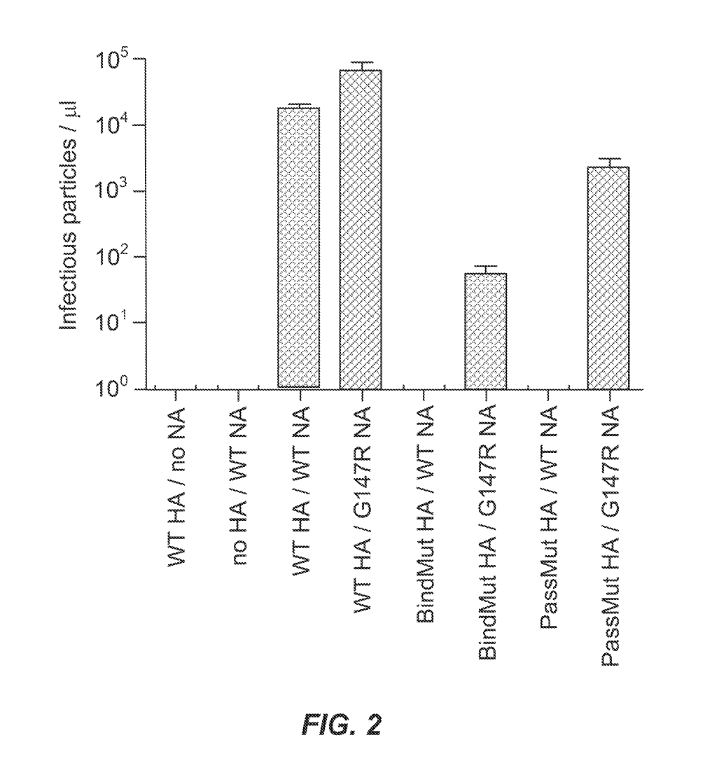

As shown in FIG. 2, influenza virus carrying the WT HA paired with either WT or G147R NA was efficiently rescued. But, BindMut or PassMut HAs paired with WT NA could not be rescued, which indicated that the mutated HAs were binding-deficient. In contrast, moderate levels of virus carrying the BindMut HA and NA^{G147R} could be rescued, which shows that NA^{G147R} compensated for the loss of HA receptor binding. Moreover, virus containing PassMut HA and NA^{G147R} grew to levels nearly as high as WT virus.

Thus, these results indicate that in the presence of a non-binding HA (HA^{nb}), the G147R mutation in NA allowed NA to provide the receptor-binding function (NA^{b}) normally provided by HA.

### Example 2

### Infection and Hemagglutination of Influenza NA Receptor Binding Mutant

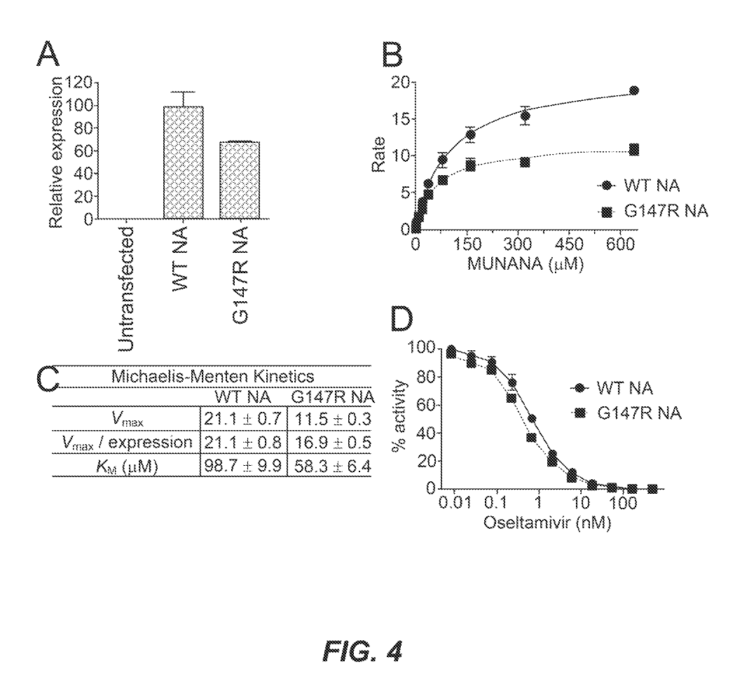

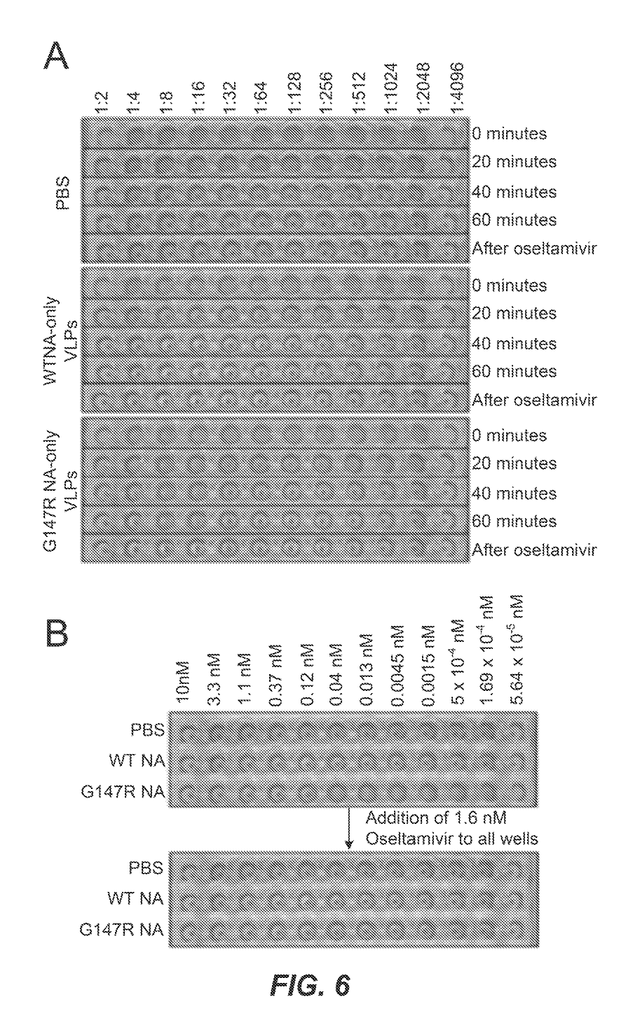

To conclusively show that the cell binding of PassMut HA/G147R NA is completely independent of HA, virus-like particles (VLPs) that expressed NA but no HA were produced. This was done by transfecting 293T cells with plasmids expressing M1 and M2 and either WT or G147R NA, as NA alone has previously been shown to be sufficient for VLP production with M1 slightly enhancing VLP release (Lai et al., J. Gen. Virol. 91:2322, 2010), and M2 is known to promote membrane scission (Rossman et al., Cell 142:902, 2010). The total NA activity in the G147R VLP supernatant was 77% that of WT NA VLP supernatant, consistent with the slightly reduced activity of G147R NA reported in FIG. 4. Concentrated VLP supernatants were used to perform a hemagglutination assay with turkey RBCs. FIG. 6A shows images of the assay taken every 20 minutes. The WT NA-only VLPs slightly increased the speed of RBC settling relative to the PBS control, suggesting that removal of cell-surface sialic acid might promote the settling of RBCs, possibly by removing negative charges from the cell surface. At high concentrations, the G147R NA-only VLPs initially slightly agglutinated the RBCs, but this agglutination soon disappeared and the RBCs settled to the bottom of the plate. But at moderate concentrations, the G147R NA-only VLPs potently agglutinated the RBCs over the full 60-minute time course. After 60 minutes, oseltamivir was added to all wells at a high concentration. Oseltamivir reversed the agglutination by the G147R VLPs, consistent with the idea that oseltamivir can elute the VLPs off the RBCs by competitively binding to the G147R NA.

Overall, the results in FIG. 6A show that the G147R NA can bind VLPs to RBCs in a reversible manner. The eventual disappearance of agglutination at high G147R NA only VLP concentrations suggests that G147R NA might slowly cleave the same receptor to which it initially binds. In this scenario, at high VLP concentrations the G147R NA eventually removes all of the receptor, making the RBCs resistant to continued agglutination. At moderate VLP concentrations, the rate of receptor removal is lower and so long-term agglutination is observed.

A hemagglutination-inhibition assay was next performed in the presence of increasing dilutions of oseltamivir and a G147R NA-only VLP concentration that caused long-term agglutination. Oseltamivir inhibited agglutination by the G147R NA-only VLPs down to concentrations of 0.12 nM. At lower oseltamivir concentrations, agglutination did occur, but it could again be reversed by the addition of high concentrations of oseltamivir after one hour (FIG. 6B).

Taken together, these data show that G147R NA is sufficient for agglutination in the complete absence of HA.

### Example 3

### HA Still Required for Viral Fusion

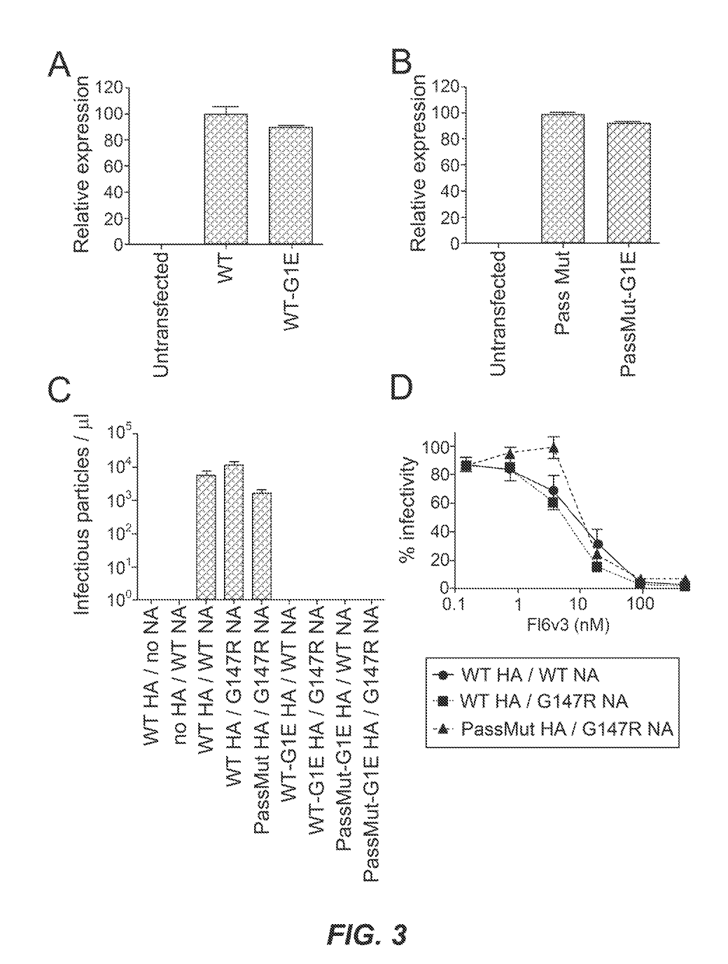

Although NA was functioning as the receptor-binding protein in the mutant viruses, it still remained to be determined whether HA was still needed to mediate membrane fusion. To test this, a point mutation that has been shown to abolish the fusion activity of HA, G1E in HA2 (Qiao et al., Mol. Biol. Cell 10:2759, 1999). The G1E mutation was introduced into both the WT and PassMut HA. To confirm that G1E did not affect HA levels at the cell surface, we used cell-surface staining with polyclonal anti-HA serum and flow cytometry to quantify cell-surface protein levels. Serum from mice infected with WT HA virus was used to stain WT and WT-G1E expressing cells, while serum from mice infected with PassMut HA virus was used to stain PassMut and PassMut-G1E expressing cells. In both cases, expression of the G1E mutant was greater than 90% that of the matched parent HA (FIGS. 3A and B), indicating that G1E does not substantially impair HA folding or trafficking to the cell surface.

Then, it was tested whether virus containing the G1E HAs with either WT or G147R NA could rescue the fusion mutant. Rescue of G1E-containing viruses was not possible, indicating that abolishing HA's fusion function ablates viral growth (FIG. 3C).

To further confirm the requirement for HA-mediated fusion, neutralization assays were performed with the anti-fusion antibody FI6v3 (Corti et al., Science 333:850, 2011). This broadly neutralizing antibody locks HA into the pre-fusion conformation. All viruses were neutralized by FI6v3 at similar concentrations, regardless of their HA and NA composition (FIG. 3D).

Taken together, these data show that HA is required for fusion regardless of whether or not the virus has NA with the G147R mutation.

### Example 4

### Examining G147R NA Receptor

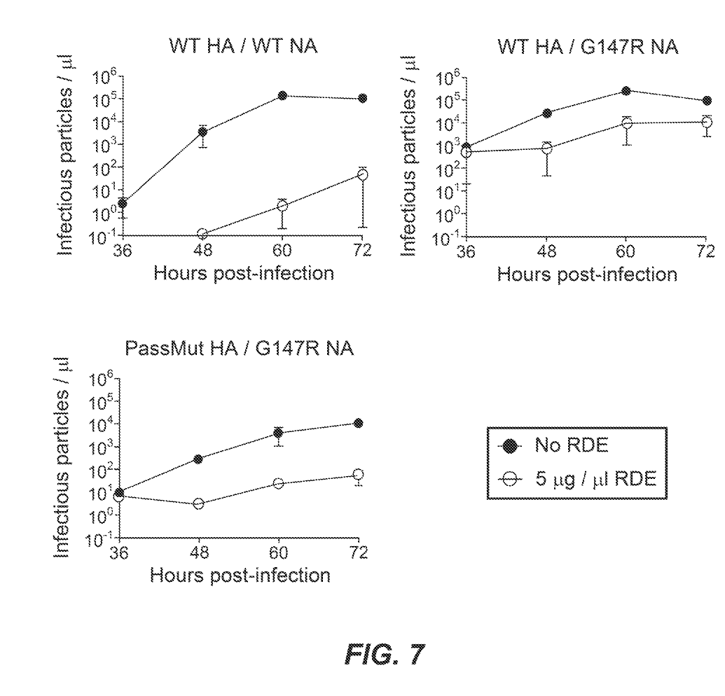

To determine whether the G147R mutant NA still binds to the canonical sialic-acid receptor recognized by HA, cells were pre-treated with a broad spectrum bacterial sialidase (receptor destroying enzyme, RDE) for one hour, and then infected with WT HA/WT NA, WT HA/G147R NA, and PassMut HA/G147R NA viruses at an MOI of 0.05. The viral supernatant was titered every 12 hours beginning at 36 hours post-infection. RDE treatment nearly completed inhibited growth of WT HA/WT NA except for low levels of viral growth at late time points (FIG. 7). However, the WT HA/G147R NA and PassMut HA/G147R NA viruses were substantially less inhibited by RDE treatment of the cells (FIG. 7), although their growth was still clearly reduced.

These results suggest that the receptor for the G147R NA is more refractory to RDE cleavage than the receptor for HA. However, it is unclear whether the G147R NA recognizes a non-sialic acid receptor, or simply recognizes a class of sialic acid moieties that is partially resistant to RDE cleavage.

### Example 5

### Effect of Oseltamivir and Anti-NA Antibodies on G147 NA Viral Infection and Hemagglutination

The ability of oseltamivir to inhibit enzyme activity for both the WT and G147R NA was tested (FIG. 4D). Both variants were inhibited by oseltamivir at similar concentrations, indicating that oseltamivir can still bind to the active site of the G147R NA. Therefore, oseltamivir was tested for its ability to inhibit the receptor binding of viruses dependent on the G147R NA.

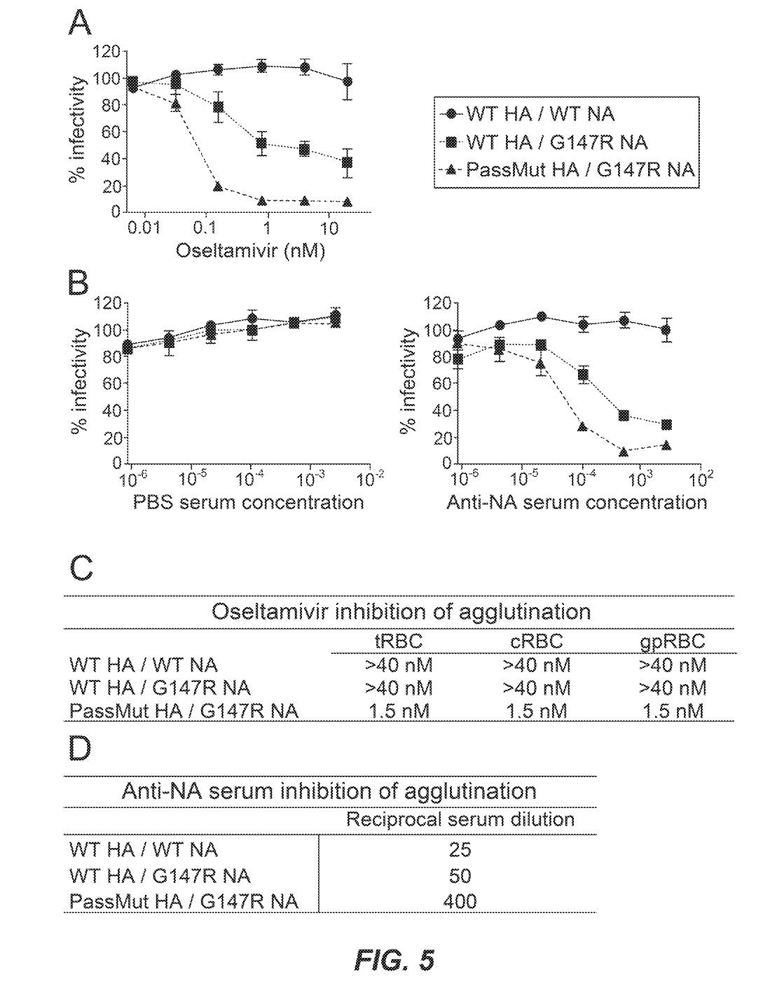

Oseltamivir's effect on infectivity was tested on three viruses: WT HA/WT NA, WT HA/G147R NA, and PassMut HA/G147R NA (FIG. 5A). WT HA/WT NA virus was uninhibited at all concentrations tested, consistent with the prevailing belief that NA activity is not crucial for viral entry (Liu et al., J. Virol. 69:1099, 1995). However, PassMut HA/G147R NA was strongly neutralized at low nanomolar oseltamivir concentrations, consistent with the results herein showing that NA is the viral attachment protein for this virus. WT HA/G147R NA showed an intermediate phenotype, likely because oseltamivir inhibits NA-mediated but not HA mediated receptor binding by this virus.

Whether polyclonal mouse serum with NA-specific antibodies could block infectivity was then tested. Serum was obtained from mice infected with a virus containing G147R NA, but an H1 subtype HA. Because the WT and G147R NAs differ at only a single site, this polyclonal serum should substantially react with both NAs, but should not recognize the H3 subtype HA present in all three viruses tested. The degree of neutralization of the three viruses by this serum was similar to that seen for oseltamivir (FIG. 5B). PassMut HA/G147R NA was strongly neutralized, WT HA/WT NA was completely uninhibited, and WT HA/G147R NA showed an intermediate phenotype.

To directly test if oseltamivir blocks viral attachment to cells, hemagglutination-inhibition assays were performed. All red blood cell (RBC) types tested (turkey, chicken, and guinea pig) were effectively agglutinated by the PassMut HA/G147R NA virus, but in all cases this agglutination was inhibited down to an oseltamivir concentration of 1.5 nM. In contrast, the WT HA/WT NA and WT HA/G147R NA were uninhibited at all oseltamivir concentrations tested (FIG. 5C).

A hemagglutination-inhibition assay was also performed in the presence of purified polyclonal anti-NA antibodies from mouse serum. PassMut HA/G147R NA was potently inhibited, while WT HA/WT NA and WT HA/G147R NA were much more resistant (FIG. 5D).

Taken together, these data show that infectivity and cell binding of PassMut HA/G147R NA virus are inhibited by blocking NA with either a small molecule inhibitor or polyclonal antibodies. These results strongly suggest that the PassMut HA/G147R NA viruses are using NA as the sole receptor-binding protein.

### Example 6

### Activity of Viruses Containing HA-Epitope Fusions

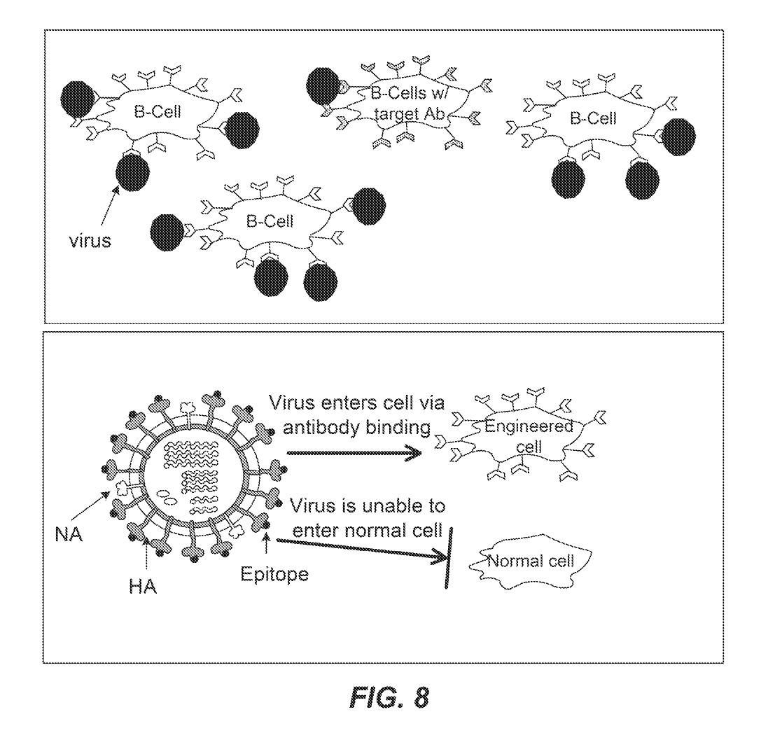

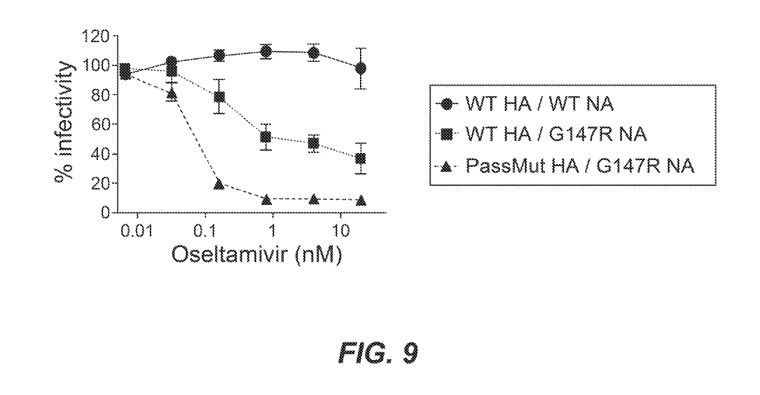

Influenza contains two surface proteins, hemagglutinin (HA) and neuraminidase (NA). Normally, HA serves as the receptor-binding protein, and is also the most immunogenic part of the virus—high levels of antibodies are elicited towards the globular head of influenza. NA normally serves as the viral release protein. A novel mutant influenza virus was engineered with extensive mutations to the receptor-binding pocket in the globular head of the HA (FIG. 1). This virus can no longer infect cells due to its mutated HA. However, another mutation in NA that allows this protein to acquire the receptor-binding activity (FIG. 8) normally performed by HA was engineered. Viruses with the mutated HA cannot grow when paired with normal NAs, but they can grow when paired with the mutant receptor binding NA (FIG. 2). Furthermore, infection of normal cells by the virus can be blocked by addition of the small molecule oseltamivir (the active compound in Tamiflu®), as shown in FIG. 9. These viruses therefore serve as an ideal platform for the approach described herein—their immunogenic HA is accommodating towards mutations in the normally conserved receptor-binding pocket, and the receptor-binding NA allows viral infection in a fashion that can be switched off by the addition of oseltamivir. These viruses can rapidly be grown to high titer using standard influenza-reverse genetics approaches (Hoffman et al., 2000), either using live influenza virus or a GFP-carrying virus that we have described previously (Bloom et al., 2010). Influenza is also naturally highly immunogenic, and elicits high levels of anti-HA antibodies in infected hosts without any need for the addition of exogenous adjuvant.

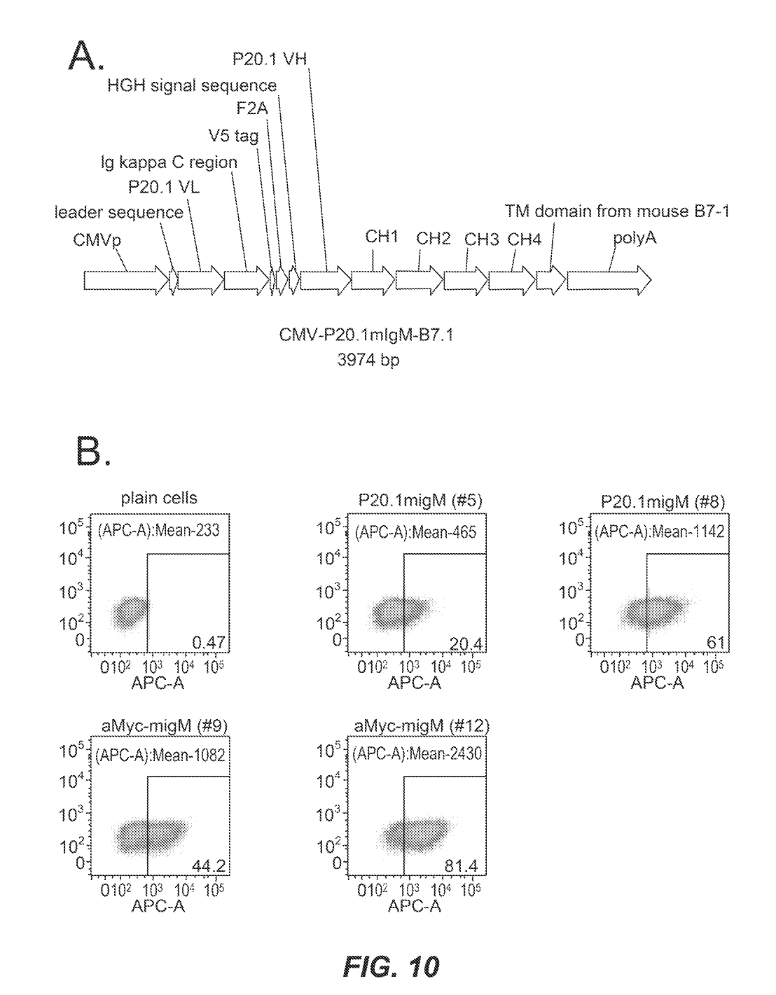

In order to make virus dependent on an antigen-antibody interaction for infection, a lentiviral construct was created to allow expression of membrane bound antibody on the surface of standard cell lines, such as 293T and MDCK cells. A schematic of this construct is shown in FIG. 10, as well as representative flow cytometry data showing how cells can be transduced to express the membrane-bound antibody on their surface.

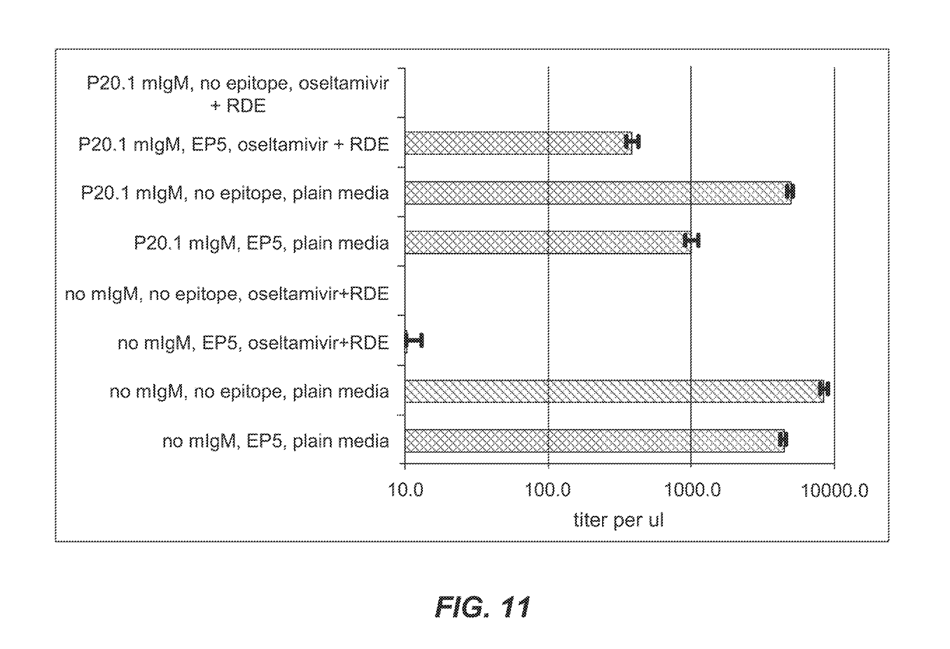

As proof of principle, the P4 antigen/P20.1 antibody pair was used (Nogi et al., Protein Sci. 17:2120, 2008). This antibody is not itself likely to be of clinical use, but provides a convenient prototype for testing the instant approach. The P4 epitope antigen is derived from the human PAR4 protein, and the P20.1 antibody recognizes this epitope. The P20.1 antibody was cloned into the construct shown in FIG. 11, and engineered a variant of MDCK cells to express this antibody on their surface.

The P4 tag was then inserted into the globular head of the binding deficient HA, and these viruses were grown using the receptor-binding NA. In the absence of any inhibitors, viruses both with and without this epitope grew to high titers in both normal cells and cells expressing the P20.1 antibody, as they could enter cells using the NA. But, when the small molecule inhibitor oseltamivir (as well as a second possible inhibitor, the bacterial sialidase RDE) was added, viruses without the epitope could not infect any of the cells. Viruses carrying the epitope were also unable to infect normal cells. But, such viruses efficiently infected cells that expressed the target antibody P20.1, and grew to high titers in these cells.

This shows that the compositions and methods of the instant disclosure can be used to engineer and select for viral antigens that potently bind to target antibodies.

### Example 7

### Materials and Methods

### Viral Strains/Genes

All HA sequences were derived from the A/Hong Kong/2/1968 (X31) H3N2 strain. Mutations to add potential glycosylation sites (Table 2) were first introduced into the parental X31 HA through site-directed mutagenesis. This HA variant is referred to as “WT” throughout these examples. Receptor-binding site mutations (Table 3) were then introduced through site-directed mutagenesis to the WT variant to create the “BindMut HA.” A third variant, named “PassMut HA” also has the additional HA-stalk mutation, K62E in HA2, introduced through site-directed mutagenesis. All NA sequences were derived from the A/WSN/33 (WSN) N1 strain. The G147R point mutation was introduced through site-directed mutagenesis. The other viral genes (PB1, PB2, PA, NP, M, NS) were also from the A/WSN/33 strain. The coding sequences for all HA and NA variants are provided herein as SEQ ID NOS.:1-5.

### Plasmids

All HA and NA variants generated during this study were cloned into the bidirectional pHW2000 backbone for reverse-genetics viral rescue (Hoffmann et al., Proc Nat'l. Acad. Sci. USA 97:6108, 2000). The other viral genes were expressed from previously described bidirectional WSN reverse-genetics plasmids (Hoffmann et al., 2000), which were a kind gift from Robert Webster of St. Jude Children's Research Hospital. For viral rescue experiments, we used a previously described GFP-based system where the coding region of PB1 is replaced by the coding region of GFP (Bloom et al., Science 328:1272, 2010). This plasmid is referred to as “PB1flank-eGFP.” For some of the experiments, HA and NA were also cloned into an expression plasmid (HDM) which places the gene under the control of a CMV promoter followed by an IRES-GFP and the beta-globin polyA element.

### Cells

Viruses carrying GFP in the PB1 segment were grown in previously described 293T and MDCK-SIAT1 cell lines that constitutively express PB1 under control of a CMV promoter (Bloom et al., Science 328:1272, 2010). These cell lines are named 293T-CMV-PB1 and MDCK-SIAT1-CMV-PB1, respectively.

### Viral Rescue

Co-cultures of 293T-CMV-PB1 and MDCK-SIAT1-CMV-PB1 cells were transfected with eight reverse-genetics plasmids encoding PB2, PA, NP, M, NS, HA, NA, and PB1flank eGFP. Cells were plated at a density of 2×10⁵293 T-CMV-PB1 and 0.25×10⁵ MDCK SIAT1-CMV-PB1 cells per well in 6-well dishes in D10 (DMEM supplemented with 10% heat-inactivated FBS, 2 mM L-glutamine, 100 U/ml of penicillin, and 100 μg/ml of streptomycin.) The next day, 250 ng of each plasmid was transfected into the cells using the BioT transfection reagent (Bioland B01-02). At 12-18 hours post-transfection the cells were washed with PBS and the media changed to Influenza Growth Media (IGM) (OptiMEM supplemented with 0.01% heat-inactivated FBS, 0.3% BSA, 100 U/ml of penicillin, 100 μg/ml of streptomycin, and 100 μg/ml calcium chloride). TPCK-trypsin was added to IGM at 3 μg/ml immediately before use. Viral supernatants were collected 72 hours post-transfection and titered.

### Viral Titering

The titer of the PB1flank-eGFP viruses was determined by flow cytometry. Briefly, MDCK-SIAT1-CMV-PB1 cells were plated at 10⁵ per well in 12-well dishes in IGM and infected 4 hours later with 1 μl, 10 μl, and 100 μl of viral supernatant. 16 hours post infection, wells with approximately 1-10% GFP positive cells were analyzed by flow cytometry to determine the fraction of cells that were GFP positive. The Poisson equation was used to convert this fraction to the initial MOI, allowing determination of the number of infectious particles in the original inoculum.

### HA Surface Expression

The G1E point mutation in HA2 was introduced into WT and PassMut HA by site directed mutagenesis, and the mutated genes were cloned into the HDM plasmid. 293T cells were transfected with plasmid encoding each of the HA variants with and without the G1E mutation in triplicate. At 20 hours post-transfection, the cells were collected and resuspended in MOPS buffered saline (MBS) (15 mM MOPS, 145 mM sodium chloride, 2.7 mM potassium chloride, and 4.0 mM calcium chloride, adjusted to pH 7.4, 2% heat-inactivated FBS added immediately before use). Heat-inactivated polyclonal serum from influenza-infected mice at a 1:200 dilution was used as the primary antibody to stain for surface HA molecules, and a goat-anti-mouse TriColor antibody (Caltag Laboratories M32006) at a 1:100 dilution was used as the secondary antibody. Cells were analyzed by flow cytometry to determine the mean fluorescent intensity (MFI) of TriColor (APC channel) among the GFP positive (transfected) cells. Reported values for each G1E mutant are normalized to the respective wild-type.

### NA Surface Expression

A C-terminal V5 epitope tag was added to both WT and G147R NA. Both genes were then cloned into the HDM plasmid, and used to transfect 293T cells. At 20 hours post-transfection, cells were collected and stained with an anti-V5 AF647-conjugated antibody (Invitrogen 45-1098) at a 1:200 dilution. Cells were analyzed by flow cytometry to determine the MFI of AF647 (APC channel) among GFP positive (transfected) cells. Reported values were normalized to the WT NA.

### MUNANA Activity Assay

NA activity was assayed using the fluorogenic 2′-(4-Methylumbelliferyl)-alpha-D-N-acetylneuraminic acid (MUNANA) substrate (Sigma M8639). 293T cells were transfected with HDM plasmid encoding each NA variant in triplicate. At 20 hours post transfection, cells were collected and diluted 1:40 in a 96-well plate such that each row contained one NA variant. Serial two-fold dilutions of MUNANA were made across each row of a Costar black flat-bottom 96-well plate. Both plates were pre-warmed to 37° C. for 20 minutes. Cells were then quickly resuspended by pipetting and added to the MUNANA plate. Fluorescent readings were taken every minute for 1 hour at an excitation wavelength of 360 nm and an emission wavelength of 448 nm. Fluorescence above background was then plotted versus time for each MUNANA concentration to determine the reaction rate. Reaction rate was then plotted against MUANANA concentration and the K_{M} and Vₘₐₓ determined by fitting Michaelis-Menten kinetics curves in GraphPad Prism 5.

### Oseltamivir Inhibition Assay

293T cells were transfected with HDM plasmids encoding WT and G147R NA in triplicate. At 20 hours post-transfection, cells were collected, diluted, and then incubated with decreasing concentrations of oseltamivir carboxylate (kindly provided by Roche) at 37° C. for 30 minutes to allow for oseltamivir binding. MUNANA was added to 300 μM and incubation continued for 45 minutes. The reaction was quenched by adding a solution of 0.153 M NaOH in 81.5% ethanol, and the signal was read as described above. Values were normalized to a no-oseltamivir control for each NA variant to determine the percent remaining activity.

### Mouse Infections

Serum for neutralization assays, hemagglutination inhibition assays, and cell-surface staining was obtained from influenza-infected mice. Mice were intranasally infected with replication-competent virus after being anesthetized with 2 mg ketamine and 0.2 mg xylazine per mouse. At three weeks post-infection, a booster infection was done using the same protocol. Mice were then euthanized and bled by cardiac puncture 4 weeks after initial infection, or 1 week after the booster. For neutralization assays, mouse serum was heat inactivated at 56° C. for 40 minutes prior to use. For hemagglutination inhibition assays, serum was heat inactivated, then antibodies were purified by Protein A column (Thermo Scientific 89952) and concentrated to the original volume prior to use.

### Neutralization Assays

Neutralization assays were performed using the PB1flank-eGFP viruses. To reduce the background media auto-fluorescence in the GFP channel, we developed a Neutralization Assay Media (NAM) consisting of Medium 199 supplemented with 0.01% heat-inactivated FBS, 0.3% BSA, 100 U/ml of penicillin, 100 μg/ml of streptomycin, 100 μg/ml calcium chloride and 25 mM HEPES. Polyclonal serum or oseltamivir was diluted down the columns of a 96-well plate in NAM and virus was added at a multiplicity of infection (MOI) that ranged from 0.1 to 0.8 for the different viruses. Plates were incubated at 37° C. for 1 hour to allow oseltamivir or antibody binding, then 4×10⁴ MDCK-SIAT1-CMV-PB1 cells were added per well. A no-serum or no-oseltamivir control row for each virus was included to give a maximum infectivity value, and a no virus control row was included to give the background fluorescence. After an 18-hour incubation, GFP fluorescence intensity was measured using an excitation wavelength of 485 nm and an emission wavelength of 515 nm (12 nm slit widths). The signal above background for each well was normalized to its respective no-oseltamivir or no-serum control; values are reported as percent infectivity remaining averaged over triplicate measurements.

### Hemagglutination Inhibition Assays

Hemagglutination inhibition assays were performed using turkey (Lampire Biological Laboratories 7249409), chicken (Innovative Research IC05-0810), or guinea pig (Innovative Research IC05-0910) red blood cells (RBCs) diluted to 0.5% in PBS. The hemagglutination titer for each virus and blood cell type was determined, then 8 HAU used for inhibition assays. 10 μl containing 8 HAU of virus was pre-incubated at 37° C. with 40 μl serum or oseltamivir for 1 hour in U-bottom plates, then 50 μl of RBCs were added. Plates were scored after 1 hour incubation at room temperature.

### VLP Production

To produce virus-like particles (VLPs) expressing NA but not HA on their surface, 293T cells in D10 were transfected with an HDM plasmid expressing M1 and M2 from the A/PR/8/34 (H1N1) strain separated by a T2A linker, and an HDM plasmid expressing either WT or G147R NA. The media was changed to IGM at 24 hours post transfection, and the VLP supernatant was collected at 72 hours post-transfection. Supernatants were clarified at 2000×g for 5 minutes to pellet cell debris. The clarified supernatant was then concentrated with a 100 kDa cut-off centrifugal concentrator. MUNANA activity of the collected VLPs was determined for equal volumes of concentrated supernatants.

### Viral Growth in the Presence of RDE

MDCK-SIAT1-CMV-PB1 cells were plated in 6-well dishes at a density of 5×10⁴ cells per well in D10. After 18 hours, the media was changed to IGM with 4 μg/ml TPCK trypsin after a PBS wash. Half of the wells also contained the bacterial sialidase RDE (Sigma C8772, 1 vial resuspended in 5 ml sterile water) added at 5 μl/ml. Plates were incubated at 37° C. for one hour to allow for RDE cleavage, then infected at an MOI of 0.05. Beginning at 24 hours post-infection, supernatant was collected and titered every 12 hours as previously described.

TABLE 1
N1 sequences with R at position 147 (N2 numbering).

| Accession Number | Strain name | Lineage |
|---|---|---|
| ABD78030 | A/South-Canterbury/59/2000 | Seasonal H1N1 |
| ABX58495 | A/Tennessee/UR06-0238/2007 | Seasonal H1N1 |
| ACY01424 | A/Hamedan/117/2007 | Seasonal H1N1 |
| ACA33659 | A/Texas/74/2007 | Seasonal H1N1 |
| ADZ53071 | A/Hong_Kong/01045/2008 | Seasonal H1N1 |
| ADP89151 | A/Thailand/Siriraj-01/2008 | Seasonal H1N1 |
| ADP89152 | A/Thailand/Siriraj-02/2008 | Seasonal H1N1 |
| ADP89155 | A/Thailand/Siriraj-05/2008 | Seasonal H1N1 |
| ACM17331 | A/Austria/404811/2008 | Seasonal H1N1 |
| ADA69512 | A/Austria/404821/2008 | Seasonal H1N1 |
| ADA69518 | A/Austria/405179/2008 | Seasonal H1N1 |
| ACI94940 | A/Austria/405109/2008 | Seasonal H1N1 |
| BAH22142 | A/Yokohama/30/2008 | Seasonal H1N1 |
| ACM90850 | A/Johannesburg/279/2008 | Seasonal H1N1 |
| ADZ53099 | A/Hong_Kong/17566/2009 | Seasonal H1N1 |
| ADC45782 | A/Niigata/08F188/2009 | Seasonal H1N1 |
| AET84319 | A/Iraq/WRAIR1683P/2009 | Seasonal H1N1 |
| ADA71159 | A/Novosibirsk/3/2009 | Seasonal H1N1 |
| ACU44027 | A/Kentucky/08/2009 | Seasonal H1N1 |
| ACU44235 | A/Kentucky/08/2009 | Seasonal H1N1 |
| ADN26074 | A/Finland/614/2009 | Pandemic H1N1 |
| AFE11259 | A/Tianjinhedong/SWL44/2011 | Pandemic H1N1 |
| AFN20030 | A/Singapore/SGH02/2011 | Pandemic H1N1 |
| ADG59204 | A/chicken/Anhui/39/2004 | Avian H5N1 |
| ADG59211 | A/chicken/Gansu/44/2004 | Avian H5N1 |
| ADB26210 | A/chicken/Nigeria/08RS848-93/2007 | Avian H5N1 |
| AFH53768 | A/chicken/Egypt/Kalyobia-18-CLEVB/2011 | Avian H5N1 |
| AGG52920 | A/chicken/Bangladesh/12VIR-7140-1/2011 | Avian H5N1 |
| AGG52921 | A/chicken/Bangladesh/12VIR-7140-2/2012 | Avian H5N1 |
| AGG52922 | A/chicken/Bangladesh/12VIR-7140-3/2012 | Avian H5N1 |
| AGG52925 | A/chicken/Bangladesh/12VIR-7140-6/2012 | Avian H5N1 |

TABLE 2
Sequential and H3 numbering of glycosylation
site mutations added to HA
Glycosylation site mutations

| | | Asn residue | Glycosylation |
|---|---|---|---|
| Sequential | Hr | (Sequential/H3) | site change |
| S61N | S45N | 61/45 | + |
| D79N | D63N | 79/63 | + |
| T99K | T83K | 97/81 | − |
| T138N | T122N | 138/122 | + |
| G140S | G124S | 138/122 | + |
| T142N | T126N | 142/126 | + |
| G151T | G135T | 149/133 | + |
| G160N | G144N | 160/144 | + |
| G162S | G146S | 160/144 | + |
| N264T | N248T | 262/246 | + |

TABLE 3
Sequential and H3 numbering of receptor-binding
site mutations made in BindMutHA
Receptor binding site mutations

| Sequential | H3 | Amino acid change |
|---|---|---|
| 114 | 98 | Y > F |
| 199 | 183 | H > F |
| 210 | 194 | L > A |
| 237-244 | 221-228 | deletion |

X31 ″WT HA″ sequence
(SEQ ID NO.: 1)
atgaagaccatcattgctttgagctacattttctgtctggctctcggcca
agaccttccaggaaatgacaacagcacagcaacgctgtgcctgggacatc
atgcggtgccaaacggaacactagtgaaaacaatcacagatgatcagatt
gaagtgactaatgctactgagctagttcagaactcctcaacggggaaaat
atgcaacaatcctcatcgaatccttgatggaataaactgcacactgatag
atgctctattgggggaccctcattgtgatgtttttcaaaatgagaaatgg
gaccttttcgttgaacgcagcaaagctttcagcaactgttacccttatga
tgtgccagattatgcctcccttaggtcactagttgcctcgtcaggcactc
tggagtttatcaatgagagtttcaattggactggggtcactcagaatggg
acaagctcagcttgcaaaaggggacctaatagcagttttttcagtagact
gaactggttgaccaaatcaggaagcacatatccagtgctgaacgtgacta
tgccaaacaatgacaattttgacaaactatacatttgggggattcaccac
ccgagcacgaaccaagaacaaaccagcctgtatgttcaagcatcagggag
agtcacagtctctaccaggagaagccagcaaactataatcccgaatatcg
ggtccagaccctgggtaaggggtctgtctagtagaataagcatctattgg
acaatagttaagccgggagacgtactggtaattaatagtactgggaacct
aatcgctcctcggggttatttcaaaatgcgcactgggaaaagctcaataa
tgaggtcagatgcacctattgatacctgtatttctgaatgcatcactcca
aatggaagcattcccaatgacaagccctttcaaaacgtaaacaagatcac
atatggagcatgccccaagtatgttaagcaaaacaccctgaagttggcaa
cagggatgcggaatgtaccagagaaacaaactagaggcctattcggcgca
atagcaggtttcatagaaaatggttgggagggaatgatagacggttggta
cggtttcaggcatcaaaattctgagggcacaggacaagcagcagatctta
aaagcactcaagcagccatcgaccaaatcaatgggaaattgaacagggta
atcgagaagacgaacgagaaattccatcaaatcgaaaaggaattctcaga
agtagaagggagaattcaggacctcgagaaatacgttgaagacactaaaa
tagatctctggtcttacaatgcggagcttcttgtcgctctggagaatcaa
catacaattgacctgactgactcggaaatgaacaagctgtttgaaaaaac
aaggaggcaactgagggaaaatgctgaagacatgggcaatggttgcttca
aaatataccacaaatgtgacaacgcttgcatagagtcaatcagaaatggg
acttatgaccatgatgtatacagagacgaagcattaaacaaccggtttca
gatcaaaggtgttgaactgaagtctggatacaaagactggatcctgtgga
tttcctttgccatatcatgctttttgctttgtgttgttttgctggggttc
atcatgtgggcctgccagagaggcaacattaggtgcaacatttgcatttg
a
″BindMut HA″ sequence
(SEQ ID NO.: 2)
atgaagaccatcattgctttgagctacattttctgtctggctctcggcca
agaccttccaggaaatgacaacagcacagcaacgctgtgcctgggacatc
atgcggtgccaaacggaacactagtgaaaacaatcacagatgatcagatt
gaagtgactaatgctactgagctagttcagagctcctcaacggggaaaat
atgcaacaatcctcatcgaatccttgatggaatagactgcacactgatag
atgctctattgggggaccctcattgtgatgtttttcaaaatgagacatgg
gaccttttcgttgaacgcagcaaagctttcagcaactgttttccttatga
tgtgccagattatgcctcccttaggtcactagttgcctcgtcaggcactc
tggagtttatcactgagggtttcacttggactggggtcactcagaatggg
ggaagcaatgcttgcaaaaggggacctggtagcggttttttcagtagact
gaactggttgaccaaatcaggaagcacatatccagtgctgaacgtgacta
tgccaaacaatgacaattttgacaaactatacatttgggggattttccac
ccgagcacgaaccaagagcaaaccagcgcgtatgttcaagcatcagggag
agtcacagtctctaccaggagaagccagcaaactataatcccgaatatcg
ggtccagaagaataagcatctattggacaatagttaagccgggagacgta
ctggtaattaatagtaatgggaacctaatcgctcctcggggttatttcaa
aatgcgcactgggaaaagctcaataatgaggtcagatgcacctattgata
cctgtatttctgaatgcatcactccaaatggaagcattcccaatgacaag
ccctttcaaaacgtaaacaagatcacatatggagcatgccccaagtatgt
taagcaaaacaccctgaagttggcaacagggatgcggaatgtaccagaga
aacaaactagaggcctattcggcgcaatagcaggtttcatagaaaatggt
tgggagggaatgatagacggttggtacggtttcaggcatcaaaattctga
gggcacaggacaagcagcagatcttaaaagcactcaagcagccatcgacc
aaatcaatgggaaattgaacagggtaatcgagaagacgaacgagaaattc
catcaaatcgaaaaggaattctcagaagtagaagggagaattcaggacct
cgagaaatacgttgaagacactaaaatagatctctggtcttacaatgcgg
agcttcttgtcgctctggagaatcaacatacaattgacctgactgactcg
gaaatgaacaagctgtttgaaaaaacaaggaggcaactgagggaaaatgc
tgaagacatgggcaatggttgcttcaaaatataccacaaatgtgacaacg
cttgcatagagtcaatcagaaatgggacttatgaccatgatgtatacaga
gacgaagcattaaacaaccggtttcagatcaaaggtgttgaactgaagtc
tggatacaaagactggatcctgtggatttcctttgccatatcatgctttt
tgctttgtgttgttttgctggggttcatcatgtgggcctgccagagaggc
aacattaggtgcaacatttgcatttga
″PassMut HA″ sequence
(SEQ ID NO.: 3)
atgaagaccatcattgctttgagctacattttctgtctggctctcggcca
agaccttccaggaaatgacaacagcacagcaacgctgtgcctgggacatc
atgcggtgccaaacggaacactagtgaaaacaatcacagatgatcagatt
gaagtgactaatgctactgagctagttcagaactcctcaacggggaaaat
atgcaacaatcctcatcgaatccttgatggaataaactgcacactgatag
atgctctattgggggaccctcattgtgatgtttttcaaaatgagaaatgg
gaccttttcgttgaacgcagcaaagctttcagcaactgttttccttatga
tgtgccagattatgcctcccttaggtcactagttgcctcgtcaggcactc
tggagtttatcaatgagagtttcaattggactggggtcactcagaatggg
acaagctcagcttgcaaaaggggacctaatagcagttttttcagtagact
gaactggttgaccaaatcaggaagcacatatccagtgctgaacgtgacta
tgccaaacaatgacaattttgacaaactatacatttgggggattttccac
ccgagcacgaaccaagagcaaaccagcgcgtatgttcaagcatcagggag
agtcacagtctctaccaggagaagccagcaaactataatcccgaatatcg
ggtccagaagaataagcatctattggacaatagttaagccgggagacgta
ctggtaattaatagtactgggaacctaatcgctcctcggggttatttcaa
aatgcgcactgggaaaagctcaataatgaggtcagatgcacctattgata
cctgtatttctgaatgcatcactccaaatggaagcattcccaatgacaag
ccctttcaaaacgtaaacaagatcacatatggagcatgccccaagtatgt
taagcaaaacaccctgaagttggcaacagggatgcggaatgtaccagaga
aacaaactagagaactattcggcgcaatagcaggtttcatagaaaatggt
tgggagggaatgatagacggttggtacggtttcaggcatcaaaattctga
gggcacaggacaagcagcagatcttaaaagcactcaagcagccatcgacc
aaatcaatgggaaattgaacagggtaatcgagaagacgaacgaggaattc
catcaaatcgaaaaggaattctcagaagtagaagggagaattcaggacct
cgagaaatacgttgaagacactaaaatagatctctggtcttacaatgcgg
agcttcttgtcgctctggagaatcaacatacaattgacctgactgactcg
gaaatgaacaagctgtttgaaaaaacaaggaggcaactgagggaaaatgc
tgaagacatgggcaatggttgcttcaaaatataccacaaatgtgacaacg
cttgcatagagtcaatcagaaatgggacttatgaccatgatgtatacaga
gacgaagcattaaacaaccggtttcagatcaaaggtgttgaactgaagtc
tggatacaaagactggatcctgtggatttcctttgccatatcatgctttt
tgctttgtgttgttttgctggggttcatcatgtgggcctgccagagaggc
aacattaggtgcaacatttgcatttg
WSN ″WT NA″ sequence
(SEQ ID NO.: 4)
atgaatccaaaccagaaaataataaccattgggtcaatctgtatggtagt
cggaataattagcctaatattgcaaataggaaatataatctcaatatgga
ttagccattcaattcaaaccggaaatcaaaaccatactggaatatgcaac
caaggcagcattacctataaagttgttgctgggcaggactcaacttcagt
gatattaaccggcaattcatctctttgtcccatccgtgggtgggctatac
acagcaaagacaatggcataagaattggttccaaaggagacgtttttgtc
ataagagagccttttatttcatgttctcacttggaatgcaggaccttttt
tctgactcaaggcgccttactgaatgacaagcattcaagggggaccttta
aggacagaagcccttatagggccttaatgagctgccctgtcggtgaagct
ccgtccccgtacaattcaaggtttgaatcggttgcttggtcagcaagtgc
atgtcatgatggaatgggctggctaacaatcggaatttctggtccagatg
atggagcagtggctgtattaaaatacaaccgcataataactgaaaccata
aaaagttggaggaagaatatattgagaacacaagagtctgaatgtacctg
tgtaaatggttcatgttttaccataatgaccgatggcccaagtgatgggc
tggcctcgtacaaaattttcaagatcgagaaggggaaggttactaaatcg
atagagttgaatgcacctaattctcactacgaggaatgttcctgttaccc
tgataccggcaaagtgatgtgtgtgtgcagagacaattggcacggttcga
accgaccatgggtgtccttcgaccaaaacctagattataaaataggatac
atctgcagtggggttttcggtgacaacccgcgtcccaaagatggaacagg
cagctgtggcccagtgtctgctgatggagcaaacggagtaaagggatttt
catataagtatggcaatggtgtttggataggaaggactaaaagtgacagt
tccagacatgggtttgagatgatttgggatcctaatggatggacagagac
tgatagtaggttctctatgagacaagatgttgtggcaataactaatcggt
cagggtacagcggaagtttcgttcaacatcctgagctaacagggctagac
tgtatgaggccttgcttctgggttgaattaatcagggggctacctgagga
ggacgcaatctggactagtgggagcatcatttctttttgtggtgtgaata
gtgatactgtagattggtcttggccagacggtgctgagttgccgttcacc
attgacaagtag
″G147R NA″ sequence
(SEQ ID NO.: 5)
atgaatccaaaccagaaaataataaccattgggtcaatctgtatggtagt
cggaataattagcctaatattgcaaataggaaatataatctcaatatgga
ttagccattcaattcaaaccggaaatcaaaaccatactggaatatgcaac
caaggcagcattacctataaagttgttgctgggcaggactcaacttcagt
gatattaaccggcaattcatctctttgtcccatccgtgggtgggctatac
acagcaaagacaatggcataagaattggttccaaaggagacgtttttgtc
ataagagagccttttatttcatgttctcacttggaatgcaggaccttttt
tctgactcaaggcgccttactgaatgacaagcattcaaggaggaccttta
aggacagaagcccttatagggccttaatgagctgccctgtcggtgaagct
ccgtccccgtacaattcaaggtttgaatcggttgcttggtcagcaagtgc
atgtcatgatggaatgggctggctaacaatcggaatttctggtccagatg
atggagcagtggctgtattaaaatacaaccgcataataactgaaaccata
aaaagttggaggaagaatatattgagaacacaagagtctgaatgtacctg
tgtaaatggttcatgttttaccataatgaccgatggcccaagtgatgggc
tggcctcgtacaaaattttcaagatcgagaaggggaaggttactaaatcg
atagagttgaatgcacctaattctcactacgaggaatgttcctgttaccc
tgataccggcaaagtgatgtgtgtgtgcagagacaattggcacggttcga
accgaccatgggtgtccttcgaccaaaacctagattataaaataggatac
atctgcagtggggttttcggtgacaacccgcgtcccaaagatggaacagg
cagctgtggcccagtgtctgctgatggagcaaacggagtaaagggatttt
catataagtatggcaatggtgtttggataggaaggactaaaagtgacagt
tccagacatgggtttgagatgatttgggatcctaatggatggacagagac
tgatagtaggttctctatgagacaagatgttgtggcaataactaatcggt
cagggtacagcggaagtttcgttcaacatcctgagctaacagggctagac
tgtatgaggccttgcttctgggttgaattaatcagggggctacctgagga
ggacgcaatctggactagtgggagcatcatttctttttgtggtgtgaata
gtgatactgtagattggtcttggccagacggtgctgagttgccgttcacc
attgacaagtag

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A recombinant Orthomyxoviridae virion, comprising a modified genome encoding a fusion protein of a non-binding hemagglutinin (HA^{nb}) variant with an epitope or an exogenous binding domain (EBD), and encoding a binding neuraminidase (NA^{b}) variant capable of binding to a target cell, wherein each Orthomyxoviridae virion displays on its surface the HA^{nb}-epitope fusion protein or the HA^{nb}-EBD fusion protein, respectively, and the NA^{b} protein.

2., 3. (canceled)

4. The recombinant Orthomyxoviridae virion according to claim 1, wherein:
(a) the HA^{nb} protein comprises a partial or complete globular head deletion, wherein the deleted HA^{nb} protein maintains a functional fusion domain;
(b) the HA^{nb} protein comprises a deletion ranging from about 10 amino acids to all amino acid residues at position 53 to 276 based on wild-type amino acid sequence numbering of influenza A subtype 3 hemagglutinin;
(c) the HA^{nb} protein comprises a deletion mutation of amino acid residues 221 to 228 based on wild-type amino acid sequence numbering of influenza A subtype 3 hemaglutinin;
(d) the HA^{nb} protein comprises a substitution mutation at position 98, 183, 194, or any combination thereof based on numbering of the wild type amino acid sequence of influenza A subtype 3 hemagglutinin;
(e) the HA^{nb} fusion protein comprises one to ten mutations that add one or more glycosylation sites; or
(f) any combination thereof.

5.-7. (canceled)

8. The recombinant Orthomyxoviridae virion according to claim 4, wherein the substitution mutations of subpart (d) are Y98F, H183F, L194A, or any combination thereof.

9. (canceled)

10. The recombinant Orthomyxoviridae virion according to claim 4, wherein the added glycosylation site mutations of subpart (e) comprise a substitution mutation at position 45, 63, 83, 122, 124, 126, 135, 144, 146, 248, or any combination thereof based on numbering of the wild type amino acid sequence of influenza A subtype 3 hemagglutinin.

11. The recombinant Orthomyxoviridae virion according to claim 10, wherein the substitution mutations are S45N, D63N, T83K, T122N, G124S, T126N, G135T, G144N, G146S, N248T, or any combination thereof.

12. The recombinant Orthomyxoviridae virion according to claim 1, wherein the NA^{b} protein comprises a substitution mutation at position 147 based on numbering of the wild type amino acid sequence of influenza A subtype 2 neuraminidase.

13. The recombinant Orthomyxoviridae virion according to claim 1, wherein the virion is based on an Influenzavirus, Isavirus, or Thogotovirus.

14. The recombinant Orthomyxoviridae virion according to claim 13, wherein the Influenzavirus is an influenza A virus, influenza B virus, or influenza C virus.

15. The recombinant Orthomyxoviridae virion according to claim 14, wherein the virion is based on an influenza A virus subtype comprising any combination of hemagglutinin and neuramindase subtypes, wherein the hemagglutinin subtype is selected from H1 to H17 and the neuramindase subtype is selected from N1 to N10.

16. The recombinant Orthomyxoviridae virion according to claim 15, wherein the influenza A virus subtype is H1N1, H1N2, H2N2, H3N2, H5N1, H5N2, H7N2, H7N3, H7N7, H9N2, or H10N7.

17. The recombinant Orthomyxoviridae virion according to claim 13, wherein the Thogotovirus is a Thogoto virus or a Dhori virus.

18. The recombinant Orthomyxoviridae virion according to claim 13, wherein the virion is based on a Quaranfil virus, a Johnston Atoll virus, a Lake Chad virus, or a Cygnet River virus.

19. The recombinant Orthomyxoviridae virion according to claim 1, wherein the epitope comprises from eight to about 500 amino acids.

20. The recombinant Orthomyxoviridae virion according to claim 1, wherein the epitope is specific for a known antibody, cell surface receptor, or cell surface protein.

21. The recombinant Orthomyxoviridae virion according to claim 20, wherein the cell surface receptor is a chimeric antigen receptor.

22. The recombinant Orthomyxoviridae virion according to claim 1, wherein the exogenous binding domain is a single chain antibody variable region, a single chain T cell receptor variable region, a receptor ectodomain, or a ligand.

23. The recombinant Orthomyxoviridae virion according to claim 22, wherein the single chain antibody variable region is a domain antibody, sFv, scTv, scFv, F(ab′)₂, or Fab.

24. The recombinant Orthomyxoviridae virion according to claim 1, wherein the Orthomyxoviridae virion genome comprises a truncated PB1 coding sequence comprising about 80 coding nucleotides of the PB1 5′-terminus and about 80 coding nucleotides of the PB1 3′-terminus flanking and fused to a reporter molecule.

25. The recombinant Orthomyxoviridae virion according to claim 24, wherein the about 80 coding nucleotides of the PB1 5′-terminus comprise mutations at each potential start codon.

26. The recombinant Orthomyxoviridae virion according to claim 24, wherein the reporter molecule is green fluorescent protein, enhanced green fluorescent protein (eGFP), red fluorescent protein, luciferase, aequorin, β-galactosidase, or alkaline phosphatase.

27.-55. (canceled)

56. A method for identifying an epitope, comprising:
(a) contacting a cell with an inhibitor of a binding neuraminidase (NA^{b}) variant from binding to an acceptor molecule on the cell and a population of Orthomyxoviridae virions comprising a modified genome containing a nucleic acid molecule that encodes a non-binding hemagglutinin variant (HA^{nb})-epitope fusion protein or a HA^{nb}-exogenous binding domain (EBD) fusion protein, and encoding the binding NA^{b} variant capable of binding to an acceptor molecule on the cell, wherein each virion displays at its surface a HA^{nb}-epitope fusion protein or HA^{nb}-EBD fusion protein, respectively, and the population of epitopes or EBDs, respectively, have a range of binding specificities, wherein at least one epitope or EBD, respectively, in the population of epitopes or EBD, respectively, is capable of specifically binding a target molecule on the cell that is not the neuraminidase acceptor molecule and is capable of promoting viral replication;
(b) detecting a virion that replicates on the cells in the presence of the inhibitor of the binding NA^{b} variant from binding, thereby identifying an epitope or EBD, respectively, with a desired specificity for the target molecule.

57.-83. (canceled)

84. A method for eliciting an immune response against an epitope, comprising administering a recombinant Orthomyxoviridae virion according to claim 1.

85. The method according to claim 84, wherein the recombinant Orthomyxoviridae virion is produced according to the method of claim 56.

86. The method according to claim 84, wherein the immune response is an antibody specific for the epitope.

87. The method according to claim 84, wherein the antibody is a neutralizing antibody.

88.-150. (canceled)
